(19) United States
(12) Patent Application Publication
(10) Pub. No.: US 2008/0199866 A1
(43) Pub. Date: Aug. 21, 2008

(54) SNP DETECTION AND OTHER METHODS FOR CHARACTERIZING AND TREATING BIPOLAR DISORDER AND OTHER AILMENTS

(75) Inventors: Huda Akil, Ann Arbor, MI (US); Stanley J. Watson, Ann Arbor, MI (US); Simon J. Evans, Milan, MI (US); Cortney Turner, Ann Arbor, MI (US); Rene Bernard, Ann Arbor, MI (US); Ilan Kerman, Ann Arbor, MI (US); Robert Thompson, Ann Arbor, MI (US); Margit Burmeister, Ann Arbor, MI (US); Laura J. Scott, Ann Arbor, MI (US); Fan Meng, Ann Arbor, MI (US); Michael Boehnke, Ann Arbor, MI (US); William Bunney, Laguna Beach, CA (US); Marquis Vawter, Laguna Niguel, CA (US); Edward Jones, Winters, CA (US); Prabhakara V. Choudary, Davis, CA (US); Richard Myers, Stanford, CA (US); Alan Schatzberg, Los Altos, CA (US); Jun Li, Ann Arbor, MI (US); Devin Absher, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Board of Trustees of the Leland Stanford Junior University, Stanford, CA (US)

(21) Appl. No.: 11/868,456

(22) Filed: Oct. 5, 2007

Related U.S. Application Data
(60) Provisional application No. 60/908,923, filed on Mar. 29, 2007.
(60) Provisional application No. 60/828,943, filed on Oct. 10, 2006.

(51) Int. Cl. C12Q 1/68

(52) U.S. Cl. 435/6

(57) ABSTRACT

The present application relates to the use of SNPs and differential exon expression to characterize, diagnose or treat bipolar disorder and other mental illnesses, such as major depressive disorder and schizophrenia.

### CROSS-REFERENCE TO RELATED APPLICATIONS

This application claims the benefit of U.S. Patent Application No. 60/828,943, filed Oct. 10, 2006, entitled “SNP Detection and Other Methods for Characterizing and Treating Bipolar Disorder and Other Ailments” and U.S. Patent Application No. 60/908,923, filed Mar. 29, 2007, both of which are incorporated by reference herein, in their entirety and for all purposes.

### STATEMENT AS TO RIGHTS TO INVENTIONS MADE UNDER FEDERALLY SPONSORED RESEARCH AND DEVELOPMENT

This invention was made with government support under Conte Center grant (NIMH) L99MH60398 and grant (NIMH) R21MH074307 awarded by the National Institute of Mental Health. The government has certain rights in the invention.

### BACKGROUND OF THE INVENTION

Clinical depression, including both bipolar disorders and major depression disorders, is a major public health problem, affecting an estimated 9.5% of the adult population of the United States each year. While it has been hypothesized that mental illness, including mood disorders such as major depression (“MDD”) and bipolar disorder (“BP”) as well as psychotic disorders such as schizophrenia, may have genetic roots, little progress has been made in identifying gene sequences and gene products that play a role in causing these disorders, as is true for many diseases with a complex genetic origin (see, e.g., Burmeister, Biol. Psychiatry 45:522-532 (1999)).

The current lack of biomarkers and the ineffectiveness and reliability of the diagnosis and rates are important issues for the treatment of mental disorders. For example, around 15% of the population suffers from MDD while approximately 1% suffers from BP disorders. Diagnosing bipolar disorder is difficult when, as sometimes occurs, the patient presents only symptoms of depression to the clinician. At least 10-15% of BP patients are reported to be misdiagnosed as MDD. The consequences of such misdiagnosis include a delay in being introduced to efficacious treatment with mood stabilizers and a delay in seeking or obtaining counseling specific to bipolar disorder. Also treatment with antidepressants alone induces rapid cycling, switching to manic or mixed state, and consequently increases the risk of suicide. Furthermore, in addition to a lack of efficacy, long onset of action and side effects (sexual, sleep, weight gain, etc.), there are recent concerns relating to the undesirable effects of antidepressants on metabolic syndromes, such as diabetes and hypercholesteremia.

Clearly, there is a need for methods of obtaining accurate and objective information about the physiological and/or genetic status of depressed or potentially suicidal patients, particularly as the patient's physiological and/or genetic status relates to the likely response of the patient to a particular treatment regimen.

### BRIEF SUMMARY OF THE INVENTION

The present application discloses an invention comprising several embodiments. For instance, in one embodiment, the invention provides a method for identifying a human subject having an increased risk of bipolar disorder, the method comprising: a) obtaining a nucleic acid sample from the subject; and b) identifying an occurrence of a single nucleotide polymorphism (SNP) selected from the SNPs of Table 1 and/or Table 2, wherein an occurrence of one or more of SNPs is associated with increased risk of developing bipolar disorder. In a related embodiment, the method further comprises recording or reporting the risk of developing bipolar disorder. In another related embodiment, the method comprises a step of reporting said result to a physician or the human subject of the analysis.

In another embodiment, the invention provides a method for identifying a human subject likely to respond to therapy for bipolar disorder, comprising: a) obtaining a nucleic acid sample from the subject; and b) identifying an occurrence of a single nucleotide polymorphism (SNP) selected from the SNPs of Table 1 and/or Table 2, wherein an occurrence of one or more of SNPs is associated with an increased likelihood of responding to therapy for bipolar disorder. In a related embodiment, the method further comprises recording or reporting the risk of developing bipolar disorder. In another related embodiment, the method comprises a step of reporting said result to a physician or the human subject of the analysis.

In yet another embodiment, the invention provides a method for diagnosing bipolar disorder in a human subject, comprising: a) obtaining a nucleic acid sample from the subject; and b) identifying an occurrence of a single nucleotide polymorphism (SNP) selected from the SNPs of Table 1 and/or Table 2, wherein an occurrence of one or more of SNPs is associated with an increased likelihood that the patient is suffering from bipolar disorder. In a related embodiment, the method further comprises recording or reporting the risk of developing bipolar disorder. In another related embodiment, the method comprises a step of reporting said result to a physician or the human subject of the analysis.

In yet another embodiment, the invention provides a method for diagnosing bipolar disorder in a human subject, comprising: a) obtaining a nucleic acid sample from the subject; and b) identifying an occurrence of a single nucleotide polymorphism (SNP) in linkage equilibrium with one or more of the SNPs or genes of Table 1 and/or Table 2, wherein an occurrence of one or more SNPs in linkage disequilibrium with an SNP or gene of Table 1 and/or Table 2 is associated with an increased likelihood that the patient is suffering from bipolar disorder.

In yet another embodiment, the invention provides method for diagnosing the presence of a polymorphism in a human gene selected from the list of genes in Table 2, wherein said polymorphism predisposes said human to bipolar disease, said method comprising: obtaining a sample from a human subject; contacting said sample with a reagent, wherein said reagent provides a detectable signal indicative of the presence of a polymorphism in said gene; and reporting or recording said diagnosis based on said signal. In a related embodiment, the polymorphism is selected from the list of polymorphisms in Table 1.

In yet another embodiment, the invention provides a kit comprising agents suitable for detecting one, two, three, four, five or more SNPs listed in Table 1 and/or Table 2, and instructions for using the agents to detect said SNPs. In a related embodiment, the agents include nucleic acid probes or primers. In another embodiment, the agents include a restriction endonuclease that discriminates between a sequence comprising an SNP of interest and one that does not contain the SNP of interest. In yet another embodiment, the kit comprises a plurality of isolated nucleic acid sequences comprising said SNPs. In yet another embodiment, the kit comprises a photograph or illustration depicting a positive detection of an SNP achieved through the use of said detection agents.

In another embodiment, the invention provides a method for identifying a human subject with an increased likelihood of schizophrenia, comprising (a) obtaining a nucleic acid sample from said subject; (b) analyzing expression of one or more of the exons in Table 3, herein; and (c) correlating a significant difference in exon expression relative to a control with an increased likelihood of schizophrenia. In a related embodiment, the exon is a differentially expressed DSC2 exon shown in FIG. 1.

In another embodiment, the invention provides a method for identifying a human subject with an increased likelihood of schizophrenia, comprising obtaining a sample from the subject; analyzing the sample for the expression of one or more of the exons of the genes listed in Table 3; correlating a significant difference in exon expression relative to a control with an increased likelihood of schizophrenia; and reporting or recording said conclusion with respect to said increased likelihood of schizophrenia. In a related embodiment, the exon is selected from the group consisting of the differentially expressed DSC2 exons, as shown in FIG. 1 and Table 7, and the differentially expressed DPM2 exon shown in Table 4 and Table 6. In another related embodiment, an SNP selected from the group consisting of SNPs rs6781 and rs7997 in exon IV of the DPM2 gene is identified in the subject and correlated with the levels of expression of said exon. In yet another related embodiment, the SNP rs12954874 in exon II of the DSC2 gene is identified in the subject and correlated with the levels of expression of said exon.

In still further embodiments related to those above, multiple biomarkers may be selected from one or more of the Tables recited in the methods, i.e., two, three, four, five or more SNPs may be selected from Table 1; or two, three, four, five or more genes (and corresponding SNPs) may be selected from Table 2; or one, two, three, four, five or more SNPs from both Tables may be selected. Multiple SNPs thus detected can provide additional diagnostic value or confirmation of a result obtained using a different SNP or set of SNPs.

Additional detail about these and other embodiments of the invention is provided by the drawings, description, tables, and claims, herein.

### BRIEF DESCRIPTION OF THE DRAWINGS

FIG. 1 shows Desmocollin (DSC2) exon expression in lymphocytes in schizophrenic individuals (lower) and non-schizophrenic controls (higher).

### DEFINITIONS

A “mental disorder” or “mental illness” or “mental disease” or “psychiatric or neuropsychiatric disease or illness or disorder” refers to mood disorders (e.g., major depression, mania, and bipolar disorders), psychotic disorders (e.g., schizophrenia, schizoaffective disorder, schizophreniform disorder, delusional disorder, brief psychotic disorder, and shared psychotic disorder), personality disorders, anxiety disorders (e.g., obsessive-compulsive disorder) as well as other mental disorders such as substance-related disorders, childhood disorders, dementia, autistic disorder, adjustment disorder, delirium, multi-infarct dementia, and Tourette's disorder as described in Diagnostic and Statistical Manual of Mental Disorders, Fourth Edition, (DSM IV). Typically, such disorders have a genetic and/or a biochemical component as well.

A “mood disorder” refers to disruption of feeling tone or emotional state experienced by an individual for an extensive period of time. Mood disorders include major depression disorder (i.e., unipolar disorder), mania, dysphoria, bipolar disorder, dysthymia, cyclothymia and many others. See, e.g., Diagnostic and Statistical Manual of Mental Disorders, Fourth Edition, (DSM IV).

“Major depression disorder,” “major depressive disorder,” or “unipolar disorder” refers to a mood disorder involving any of the following symptoms: persistent sad, anxious, or “empty” mood; feelings of hopelessness or pessimism; feelings of guilt, worthlessness, or helplessness; loss of interest or pleasure in hobbies and activities that were once enjoyed, including sex; decreased energy, fatigue, being “slowed down”; difficulty concentrating, remembering, or making decisions; insomnia, early-morning awakening, or oversleeping; appetite and/or weight loss or overeating and weight gain; thoughts of death or suicide or suicide attempts; restlessness or irritability; or persistent physical symptoms that do not respond to treatment, such as headaches, digestive disorders, and chronic pain. Various subtypes of depression are described in, e.g., DSM IV.

“Bipolar disorder” is a mood disorder characterized by alternating periods of extreme moods. A person with bipolar disorder experiences cycling of moods that usually swing from being overly elated or irritable (mania) to sad and hopeless (depression) and then back again, with periods of normal mood in between. Diagnosis of bipolar disorder is described in, e.g., DSM IV. Bipolar disorders include bipolar disorder I (mania with or without major depression) and bipolar disorder II (hypomania with major depression), see, e.g., DSM IV.

“A psychotic disorder” refers to a condition that affects the mind, resulting in at least some loss of contact with reality. Symptoms of a psychotic disorder include, e.g., hallucinations, changed behavior that is not based on reality, delusions and the like. See, e.g., DSM IV. Schizophrenia, schizoaffective disorder, schizophreniform disorder, delusional disorder, brief psychotic disorder, substance-induced psychotic disorder, and shared psychotic disorder are examples of psychotic disorders.

“Schizophrenia” refers to a psychotic disorder involving a withdrawal from reality by an individual. Symptoms comprise for at least a part of a month two or more of the following symptoms: delusions (only one symptom is required if a delusion is bizarre, such as being abducted in a space ship from the sun); hallucinations (only one symptom is required if hallucinations are of at least two voices talking to one another or of a voice that keeps up a running commentary on the patient's thoughts or actions); disorganized speech (e.g., frequent derailment or incoherence); grossly disorganized or catatonic behavior; or negative symptoms, i.e., affective flattening, alogia, or avolition. Schizophrenia encompasses disorders such as, e.g., schizoaffective disorders. Diagnosis of schizophrenia is described in, e.g., DSM IV. Types of schizophrenia include, e.g., paranoid, disorganized, catatonic, undifferentiated, and residual.

An “antidepressant” refers to an agents typically used to treat clinical depression. Antidepressants includes compounds of different classes including, for example, specific serotonin reuptake inhibitors (e.g., fluoxetine), tricyclic antidepressants (e.g., desipramine), and dopamine reuptake inhibitors (e.g, bupropion). Typically, antidepressants of different classes exert their therapeutic effects via different biochemical pathways. Often these biochemical pathways overlap or intersect. Additional diseases or disorders often treated with antidepressants include, chronic pain, anxiety disorders, and hot flashes.

An “agonist” refers to an agent that binds to a polypeptide or polynucleotide of the invention, stimulates, increases, activates, facilitates, enhances activation, sensitizes or up regulates the activity or expression of a polypeptide or polynucleotide of the invention.

An “antagonist” refers to an agent that inhibits expression of a polypeptide or polynucleotide of the invention or binds to, partially or totally blocks stimulation, decreases, prevents, delays activation, inactivates, desensitizes, or down regulates the activity of a polypeptide or polynucleotide of the invention.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators. Inhibitors are agents that, e.g., inhibit expression of a polypeptide or polynucleotide of the invention or bind to, partially or totally block stimulation or enzymatic activity, decrease, prevent, delay activation, inactivate, desensitize, or down regulate the activity of a polypeptide or polynucleotide of the invention, e.g., antagonists. Activators are agents that, e.g., induce or activate the expression of a polypeptide or polynucleotide of the invention or bind to, stimulate, increase, open, activate, facilitate, enhance activation or enzymatic activity, sensitize or up regulate the activity of a polypeptide or polynucleotide of the invention, e.g., agonists. Modulators include naturally occurring and synthetic ligands, antagonists, agonists, small chemical molecules and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide of the invention that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of a polypeptide or polynucleotide of the invention relative to the control is about 80%, optionally 50% or 25-1%. Activation is achieved when the activity value of a polypeptide or polynucleotide of the invention relative to the control is 110%, optionally 150%, optionally 200-500%, or 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

An “siRNA” or “RNAi” refers to a nucleic acid that forms a double stranded RNA, which double stranded RNA has the ability to reduce or inhibit expression of a gene or target gene when the siRNA expressed in the same cell as the gene or target gene. “siRNA” or “RNAi” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

The term “Table #” when used in the specification includes all sub-tables of the Table referred to unless otherwise indicated.

“Determining the functional effect” refers to assaying for a compound that increases or decreases a parameter that is indirectly or directly under the influence of a polynucleotide or polypeptide of the invention (such as assaying for a compound that affects the expression of one of the exons listed in Table 3), e.g., measuring physical and chemical or phenotypic effects. Such functional effects can be measured by any means known to those skilled in the art, e.g., changes in spectroscopic (e.g., fluorescence, absorbance, refractive index), hydrodynamic (e.g., shape), chromatographic, or solubility properties for the protein; measuring inducible markers or transcriptional activation of the protein; measuring binding activity or binding assays, e.g. binding to antibodies; measuring changes in ligand binding affinity; measurement of calcium influx; measurement of the accumulation of an enzymatic product of a polypeptide of the invention or depletion of an substrate; measurement of changes in protein levels of a polypeptide of the invention; measurement of RNA stability; G-protein binding; GPCR phosphorylation or dephosphorylation; signal transduction, e.g., receptor-ligand interactions, second messenger concentrations (e.g., cAMP, IP3, or intracellular Ca²⁺); identification of downstream or reporter gene expression (CAT, luciferase, β-gal, GFP and the like), e.g., via chemiluminescence, fluorescence, colorimetric reactions, antibody binding, inducible markers, and ligand binding assays.

Samples or assays comprising a nucleic acid or protein disclosed herein that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition is achieved when the activity value relative to the control is about 80%, preferably 50%, more preferably 25-0%. Activation is achieved when the activity value relative to the control (untreated with activators) is 110%, more preferably 150%, more preferably 200-500% (i.e., two to five fold higher relative to the control), more preferably 1000-3000% higher.

“Biological sample” includes sections of tissues such as biopsy and autopsy samples, and frozen sections taken for histologic purposes. Such samples include blood, sputum, tissue, lysed cells, brain biopsy, cultured cells, e.g., primary cultures, explants, and transformed cells, stool, urine, etc. A biological sample is typically obtained from a eukaryotic organism, most preferably a mammal such as a primate, e.g., chimpanzee or human; cow; dog; cat; a rodent, e.g., guinea pig, rat, mouse; rabbit; or a bird; reptile; or fish.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

The terms “peptidomimetic” and “mimetic” refer to a synthetic chemical compound that has substantially the same structural and functional characteristics of the polynucleotides, polypeptides, antagonists or agonists of the invention. Peptide analogs are commonly used in the pharmaceutical industry as non-peptide drugs with properties analogous to those of the template peptide. These types of non-peptide compound are termed “peptide mimetics” or “peptidomimetics” (Fauchere, Adv. Drug Res. 15:29 (1986); Veber and Freidinger TINS p. 392 (1985); and Evans et al., J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as a CCX CKR, but have one or more peptide linkages optionally replaced by a linkage selected from the group consisting of, e.g., —CH₂NH—, —CH₂S—, —CH₂—CH₂—, —CH═CH— (cis and trans), —COCH₂—, —CH(OH)CH₂—, and —CH₂SO—. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out the binding or enzymatic activities of a polypeptide or polynucleotide of the invention or inhibiting or increasing the enzymatic activity or expression of a polypeptide or polynucleotide of the invention.

The term “gene” means the segment of DNA involved in producing a polypeptide chain; it includes regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

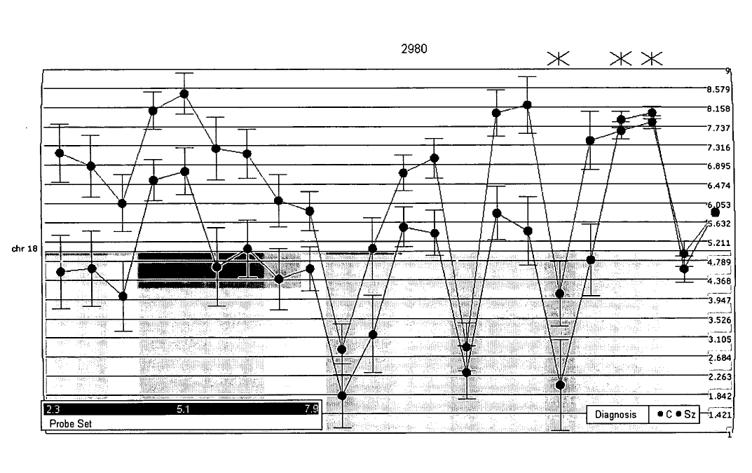

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 65° C. Such washes can be performed for 5, 15, 30, 60, 120, or more minutes. Nucleic acids that hybridize to the genes referenced in Tables 1-7 and FIG. 1 are encompassed by the invention.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications (1990).

The phrase “a nucleic acid sequence encoding” refers to a nucleic acid that contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences which may be introduced to conform with codon preference in a specific host cell.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein having an amino acid sequence encoded by any of the polynucleotides of the invention can be selected to obtain antibodies specifically immunoreactive with that protein and not with other proteins, except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

One who is “predisposed for a mental disorder” as used herein means a person who has an inclination or a higher likelihood of developing a mental disorder when compared to an average person in the general population.

“Polymorphism” refers to the occurrence of two or more genetically determined alternative sequences or alleles in a population. A “polymorphic site” refers to the locus at which divergence occurs. Preferred polymorphic sites have at least two alleles, each occurring at frequency of greater than 1%, and more preferably greater than 10% or 20% of a selected population. A polymorphic locus can be as small as one base pair (single nucleotide polymorphism, or SNP). Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allele is arbitrarily designated as the reference allele and other alleles are designated as alternative or “variant alleles.” The allele occurring most frequently in a selected population is sometimes referred to as the “wild-type” allele. Diploid organisms may be homozygous or heterozygous for the variant alleles. The variant allele may or may not produce an observable physical or biochemical characteristic (“phenotype”) in an individual carrying the variant allele. For example, a variant allele may alter the enzymatic activity of a protein encoded by a gene of interest.

The term “linkage disequilibrium” (or “LD”) refers to a situation where a particular combination of alleles (i.e., a variant form of a given gene) or a combination of polymorphisms at two loci appears more frequently than would be expected by chance. In various embodiments of the invention, significant linkage disequilibrium between an SNP and a particular variant (or variants) indicate that patients possessing such that variant (or variants) may be at risk of bipolar disease. Especially preferred are variants in significant LD with an SNP listed in Tables 1 and/or Table 2, e.g., where r²>0.3 or D′>0.75.

The term “genotype” as used herein broadly refers to the genetic composition of an organism, including, for example, whether a diploid organism is heterozygous or homozygous for one or more variant alleles of interest.

### DETAILED DESCRIPTION OF THE INVENTION

### I. Introduction

To understand the genetic basis of mental disorders, studies have been conducted to investigate SNPs and expression patterns of genes that are differentially expressed specifically in central nervous system of subjects with mood disorders. Differential and unique expression of known and novel genes was determined by way of interrogating total RNA samples purified from postmortem brains of schizophrenic patients with Affymetrix Gene Chips® (containing high-density oligonucleotide probe set arrays). SNPs associated with bipolar illness were identified by whole genome and candidate gene approaches, utilizing a large population of samples from well-characterized repositories.

The invention therefore provides methods of diagnosing mental disorders by detecting the altered expression (either higher or lower expression as indicated herein) and in some cases unique differential expression of exons referenced in Table 3 and FIG. 1 at the mRNA level in selected brain regions of patients diagnosed with mood disorders (e.g., schizophrenia) in comparison with normal individuals.

The invention further provides methods of identifying a compound useful for the treatment of such disorders by selecting compounds that modulates the functional effect of the translation products or the expression of the transcripts described herein. The invention also provides for methods of treating patients with such mental disorders, e.g., by administering the compounds of the invention or by gene therapy.

The genes and the polypeptides that they encode, which are associated with mood disorders such as bipolar disease and major depression, are useful for facilitating the design and development of various molecular diagnostic tools such as GeneChips™ containing probe sets specific for all or selected mental disorders, including but not limited to mood disorders, and as an ante-and/or post-natal diagnostic tool for screening newborns in concert with genetic counseling. Other diagnostic applications include evaluation of disease susceptibility, prognosis, and monitoring of disease or treatment process, as well as providing individualized medicine via predictive drug profiling systems, e.g., by correlating specific genomic motifs with the clinical response of a patient to individual drugs.

In addition, the present invention is useful for multiplex SNP and haplotype profiling, including but not limited to the identification of therapeutic, diagnostic, and pharmacogenetic targets at the gene, mRNA, protein, and pathway level. Profiling of splice variants and deletions is also useful for diagnostic and therapeutic applications. In particular, the SNPs referenced in Table 1 and Table 2, as well as SNPs in linkage disequilibrium with the referenced SNPs, may be identified in a test subject and used to diagnose bipolar disorder in that subject. The genes in Table 2 are deemed to be especially informative locations of SNPs for this purpose.

The genes and the polypeptides that they encode, described herein, are also useful as drug targets for the development of therapeutic drugs for the treatment or prevention of mental disorders, including but not limited to mood disorders.

Antidepressants belong to different classes, e.g., desipramine, bupropion, and fluoxetine are in general equally effective for the treatment of clinical depression, but act by different mechanisms. The similar effectiveness of the drugs for treatment of mood disorders suggests that they act through a presently unidentified common pathway. Animal models of depression, including treatment of animals with known therapeutics such as SSRIs, can be used to examine the mode of action of the genes of the invention. Lithium is the drug of choice for treating BP.

The genes and the polypeptides that they encode, described herein, as also useful as drug targets for the development of therapeutic drugs for the treatment or prevention of mental disorders, including but not limited to mood disorders. Mental disorders have a high co-morbidity with other neurological disorders, such as Parkinson's disease or Alzheimer's. Therefore, the present invention can be used for diagnosis and treatment of patients with multiple disease states that include a mental disorder such as a mood disorder. These mood disorders include BP, MDD, and other disorders such as psychotic-depression, depression and anxiety features, melancholic depression, chronic depression, BPI and BPII.

II. General Recombinant Nucleic Acid Methods for Use with the Invention

In numerous embodiments of the present invention, polynucleotides of the invention will be isolated and cloned using recombinant methods. Such polynucleotides include, e.g., those listed in Tables 1-3, which can be used for, e.g., protein expression or during the generation of variants, derivatives, expression cassettes, to monitor gene expression, for the isolation or detection of sequences of the invention in different species, for diagnostic purposes in a patient, e.g., to detect mutations or to detect expression levels of nucleic acids or polypeptides of the invention. In some embodiments, the sequences of the invention are operably linked to a heterologous promoter. In one embodiment, the nucleic acids of the invention are from any mammal, including, in particular, e.g., a human, a mouse, a rat, a primate, etc.

### A. General Recombinant Nucleic Acids Methods

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

### B. Cloning Methods for the Isolation of Nucleotide Sequences Encoding Desired Proteins

In general, the nucleic acids encoding the subject proteins are cloned from DNA sequence libraries that are made to encode cDNA or genomic DNA. The particular sequences can be located by hybridizing with an oligonucleotide probe, the sequence of which can be derived from the sequences of the genes listed in Tables 1-3, which provide a reference for PCR primers and defines suitable regions for isolating specific probes. Alternatively, where the sequence is cloned into an expression library, the expressed recombinant protein can be detected immunologically with antisera or purified antibodies made against a polypeptide comprising an amino acid sequence encoded by a gene listed in Tables 1-3.

Methods for making and screening genomic and cDNA libraries are well known to those of skill in the art (see, e.g., Gubler and Hoffman Gene 25:263-269 (1983); Benton and Davis Science, 196:180-182 (1977); and Sambrook, supra). Brain cells are an example of suitable cells to isolate RNA and cDNA sequences of the invention.

Briefly, to make the cDNA library, one should choose a source that is rich in mRNA. The mRNA can then be made into cDNA, ligated into a recombinant vector, and transfected into a recombinant host for propagation, screening and cloning. For a genomic library, the DNA is extracted from a suitable tissue and either mechanically sheared or enzymatically digested to yield fragments of preferably about 5-100 kb. The fragments are then separated by gradient centrifugation from undesired sizes and are constructed in bacteriophage lambda vectors. These vectors and phage are packaged in vitro, and the recombinant phages are analyzed by plaque hybridization. Colony hybridization is carried out as generally described in Grunstein et al., Proc. Natl. Acad. Sci. USA., 72:3961-3965 (1975).

An alternative method combines the use of synthetic oligonucleotide primers with polymerase extension on an mRNA or DNA template. Suitable primers can be designed from specific sequences of the invention. This polymerase chain reaction (PCR) method amplifies the nucleic acids encoding the protein of interest directly from mRNA, cDNA, genomic libraries or cDNA libraries. Restriction endonuclease sites can be incorporated into the primers. Polymerase chain reaction or other in vitro amplification methods may also be useful, for example, to clone nucleic acids encoding specific proteins and express said proteins, to synthesize nucleic acids that will be used as probes for detecting the presence of mRNA encoding a polypeptide of the invention in physiological samples, for nucleic acid sequencing, or for other purposes (see, U.S. Pat. Nos. 4,683,195 and 4,683,202). Genes amplified by a PCR reaction can be purified from agarose gels and cloned into an appropriate vector.

Appropriate primers and probes for identifying polynucleotides of the invention from mammalian tissues can be derived from the sequences provided herein. For a general overview of PCR, see, Innis et al. PCR Protocols: A Guide to Methods and Applications, Academic Press, San Diego (1990).

Synthetic oligonucleotides can be used to construct genes. This is done using a series of overlapping oligonucleotides, usually 40-120 bp in length, representing both the sense and anti-sense strands of the gene. These DNA fragments are then annealed, ligated and cloned.

A gene encoding a polypeptide of the invention can be cloned using intermediate vectors before transformation into mammalian cells for expression. These intermediate vectors are typically prokaryote vectors or shuttle vectors. The proteins can be expressed in either prokaryotes, using standard methods well known to those of skill in the art, or eukaryotes as described infra.

### III. Purification of Proteins of the Invention

Either naturally occurring or recombinant polypeptides of the invention can be purified for use in functional assays. Naturally occurring polypeptides, e.g., polypeptides encoded by genes listed in Tables 1-3, can be purified, for example, from mouse or human tissue such as brain or any other source of an ortholog. Recombinant polypeptides can be purified from any suitable expression system.

The polypeptides of the invention may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant polypeptides are purified. For example, proteins having established molecular adhesion properties can be reversible fused to polypeptides of the invention. With the appropriate ligand, the polypeptides can be selectively adsorbed to a purification column and then freed from the column in a relatively pure form. The fused protein is then removed by enzymatic activity. Finally the polypeptide can be purified using immunoaffinity columns.

A. Purification of Proteins from Recombinant Bacteria

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook et al., both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify proteins from bacteria periplasm. Where the protein is exported into the periplasm of the bacteria, the periplasmic fraction of the bacteria can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### B. Standard Protein Separation Techniques For Purifying Proteins

### 1. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

### 2. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

### 3. Column Chromatography

The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

It will be apparent to one of skill that chromatographic techniques can be performed at any scale and using equipment from many different manufacturers (e.g., Pharmacia Biotech).

### IV. Detection of Gene Expression

Those of skill in the art will recognize that detection of expression of polynucleotides of the invention has many uses. For example, as discussed herein, detection of the level of polypeptides or polynucleotides of the invention in a patient is useful for diagnosing mood disorders or psychotic disorders or a predisposition for a mood disorder or psychotic disorders. Moreover, detection of gene expression is useful to identify modulators of expression of the polypeptides or polynucleotides of the invention.

A variety of methods of specific DNA and RNA measurement using nucleic acid hybridization techniques are known to those of skill in the art (see, Sambrook, supra). Some methods involve an electrophoretic separation (e.g., Southern blot for detecting DNA, and Northern blot for detecting RNA), but measurement of DNA and RNA can also be carried out in the absence of electrophoretic separation (e.g., by dot blot). Southern blot of genomic DNA (e.g., from a human) can be used for screening for restriction fragment length polymorphism (RFLP) to detect the presence of a genetic disorder affecting a polypeptide of the invention.

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins Nucleic Acid Hybridization, A Practical Approach, IRL Press (1985); Gall and Pardue, Proc. Natl. Acad. Sci. U.S.A., 63:378-383 (1969); and John et al. Nature, 223:582-587 (1969).

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays,” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands that bind to labeled antibodies, fluorophores, chemiluminescent agents, enzymes, and antibodies which can serve as specific binding pair members for a labeled ligand. An introduction to labels, labeling procedures and detection of labels is found in Polak and Van Noorden Introduction to Immunocytochemistry, 2nd ed., Springer Verlag, N.Y. (1997); and in Haugland Handbook of Fluorescent Probes and Research Chemicals, a combined handbook and catalogue Published by Molecular Probes, Inc. (1996).

In general, a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

Most typically, the amount of RNA is measured by quantifying the amount of label fixed to the solid support by binding of the detection reagent. Typically, the presence of a modulator during incubation will increase or decrease the amount of label fixed to the solid support relative to a control incubation which does not comprise the modulator, or as compared to a baseline established for a particular reaction type. Means of detecting and quantifying labels are well known to those of skill in the art.

In preferred embodiments, the target nucleic acid or the probe is immobilized on a solid support. Solid supports suitable for use in the assays of the invention are known to those of skill in the art. As used herein, a solid support is a matrix of material in a substantially fixed arrangement.

A variety of automated solid-phase assay techniques are also appropriate. For instance, very large scale immobilized polymer arrays (VLSIPS™), available from Affymetrix, Inc. (Santa Clara, Calif.) can be used to detect changes in expression levels of a plurality of genes involved in the same regulatory pathways simultaneously. See, Tijssen, supra., Fodor et al. (1991) Science, 251: 767-777; Sheldon et al. (1993) Clinical Chemistry 39(4): 718-719, and Kozal et al. (1996) Nature Medicine 2(7): 753-759.

Detection can be accomplished, for example, by using a labeled detection moiety that binds specifically to duplex nucleic acids (e.g., an antibody that is specific for RNA-DNA duplexes). One preferred example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski (1986) et al. J. Immunol. Methods 89:123-130; Prooijen-Knegt (1982) Exp. Cell Res. 141:397-407; Rudkin (1976) Nature 265:472-473, Stollar (1970) Proc. Nat'l Acad. Sci. USA 65:993-1000; Ballard (1982) Mol. Immunol. 19:793-799; Pisetsky and Caster (1982) Mol. Immunol. 19:645-650; Viscidi et al. (1988) J. Clin. Microbial. 41:199-209; and Kiney et al. (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies that are commercially or publicly available. In addition to the art referenced above, general methods for producing polyclonal and monoclonal antibodies are known to those of skill in the art (see, e.g., Paul (3rd ed.) Fundamental Immunology Raven Press, Ltd., NY (1993); Coligan Current Protocols in Immunology Wiley/Greene, NY (1991); Harlow and Lane Antibodies: A Laboratory Manual Cold Spring Harbor Press, NY (1988); Stites et al. (eds.) Basic and Clinical Immunology (4th ed.) Lange Medical Publications, Los Altos, Calif., and references cited therein; Goding Monoclonal Antibodies: Principles and Practice (2d ed.) Academic Press, New York, N.Y., (1986); and Kohler and Milstein Nature 256: 495-497 (1975)). Other suitable techniques for antibody preparation include selection of libraries of recombinant antibodies in phage or similar vectors (see, Huse et al. Science 246:1275-1281 (1989); and Ward et al. Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through use of a nucleic acid amplification system that multiplies the target nucleic acid being detected. Examples of such systems include the polymerase chain reaction (PCR) system, in particular RT-PCR or real time PCR, and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferentially human cells from the cerebellum or the hippocampus, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

### V. Immunological Detection of the Polypeptides of the Invention

In addition to the detection of polynucleotide expression using nucleic acid hybridization technology, one can also use immunoassays to detect polypeptides of the invention. Immunoassays can be used to qualitatively or quantitatively analyze polypeptides. A general overview of the applicable technology can be found in Harlow & Lane, Antibodies: A Laboratory Manual (1988).

### A. Antibodies to Target Polypeptides or Other Immunogens

Methods for producing polyclonal and monoclonal antibodies that react specifically with a protein of interest or other immunogen are known to those of skill in the art (see, e.g., Coligan, supra; and Harlow and Lane, supra; Stites et al., supra and references cited therein; Goding, supra; and Kohler and Milstein Nature, 256:495-497 (1975)). Such techniques include antibody preparation by selection of antibodies from libraries of recombinant antibodies in phage or similar vectors (see, Huse et al., supra; and Ward et al., supra). For example, in order to produce antisera for use in an immunoassay, the protein of interest or an antigenic fragment thereof, is isolated as described herein. For example, a recombinant protein is produced in a transformed cell line. An inbred strain of mice or rabbits is immunized with the protein using a standard adjuvant, such as Freund's adjuvant, and a standard immunization protocol. Alternatively, a synthetic peptide derived from the sequences disclosed herein and conjugated to a carrier protein can be used as an immunogen.

Polyclonal sera are collected and titered against the immunogen in an immunoassay, for example, a solid phase immunoassay with the immunogen immobilized on a solid support. Polyclonal antisera with a titer of 10⁴ or greater are selected and tested for their cross-reactivity against unrelated proteins or even other homologous proteins from other organisms, using a competitive binding immunoassay. Specific monoclonal and polyclonal antibodies and antisera will usually bind with a K_{D} of at least about 0.1 mM, more usually at least about 1 μM, preferably at least about 0.1 μM or better, and most preferably, 0.01 μM or better.

A number of proteins of the invention comprising immunogens may be used to produce antibodies specifically or selectively reactive with the proteins of interest. Recombinant protein is the preferred immunogen for the production of monoclonal or polyclonal antibodies. Naturally occurring protein, such as one comprising an amino acid sequence encoded by a gene referenced in Table 1-4, may also be used either in pure or impure form. Synthetic peptides made using the protein sequences described herein may also be used as an immunogen for the production of antibodies to the protein. Recombinant protein can be expressed in eukaryotic or prokaryotic cells and purified as generally described supra. The product is then injected into an animal capable of producing antibodies. Either monoclonal or polyclonal antibodies may be generated for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519 (1976)). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., supra.

Once target protein specific antibodies are available, the protein can be measured by a variety of immunoassay methods with qualitative and quantitative results available to the clinician. For a review of immunological and immunoassay procedures in general see, Stites, supra. Moreover, the immunoassays of the present invention can be performed in any of several configurations, which are reviewed extensively in Maggio Enzyme Immunoassay, CRC Press, Boca Raton, Fla. (1980); Tijssen, supra; and Harlow and Lane, supra.

Immunoassays to measure target proteins in a human sample may use a polyclonal antiserum that was raised to the protein (e.g., one has an amino acid sequence encoded by a gene referenced in Tables 1-4) or a fragment thereof. This antiserum is selected to have low cross-reactivity against different proteins and any such cross-reactivity is removed by immunoabsorption prior to use in the immunoassay.

### B. Immunological Binding Assays

In a preferred embodiment, a protein of interest is detected and/or quantified using any of a number of well-known immunological binding assays (see, e.g., U.S. Pat. Nos. 4,366,241; 4,376,110; 4,517,288; and 4,837,168). For a review of the general immunoassays, see also Asai Methods in Cell Biology Volume 37: Antibodies in Cell Biology, Academic Press, Inc. NY (1993); Stites, supra. Immunological binding assays (or immunoassays) typically utilize a “capture agent” to specifically bind to and often immobilize the analyte (in this case a polypeptide of the present invention or antigenic subsequences thereof). The capture agent is a moiety that specifically binds to the analyte. In a preferred embodiment, the capture agent is an antibody that specifically binds, for example, a polypeptide of the invention. The antibody may be produced by any of a number of means well known to those of skill in the art and as described above.

Immunoassays also often utilize a labeling agent to specifically bind to and label the binding complex formed by the capture agent and the analyte. The labeling agent may itself be one of the moieties comprising the antibody/analyte complex. Alternatively, the labeling agent may be a third moiety, such as another antibody, that specifically binds to the antibody/protein complex.

In a preferred embodiment, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111: 1401-1406 (1973); and Akerstrom, et al. J. Immunol., 135:2589-2542 (1985)).

Throughout the assays, incubation and/or washing steps may be required after each combination of reagents. Incubation steps can vary from about 5 seconds to several hours, preferably from about 5 minutes to about 24 hours. The incubation time will depend upon the assay format, analyte, volume of solution, concentrations, and the like. Usually, the assays will be carried out at ambient temperature, although they can be conducted over a range of temperatures, such as 10° C. to 40° C.

### 1. Non-Competitive Assay Formats

Immunoassays for detecting proteins of interest from tissue samples may be either competitive or noncompetitive. Noncompetitive immunoassays are assays in which the amount of captured analyte (in this case the protein) is directly measured. In one preferred “sandwich” assay, for example, the capture agent (e.g., antibodies specific for a polypeptide encoded by a gene listed in Tables 1-4) can be bound directly to a solid substrate where it is immobilized. These immobilized antibodies then capture the polypeptide present in the test sample. The polypeptide thus immobilized is then bound by a labeling agent, such as a second antibody bearing a label. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

### 2. Competitive Assay Formats

In competitive assays, the amount of analyte (such as a polypeptide encoded by a gene listed in Table 1-4) present in the sample is measured indirectly by measuring the amount of an added (exogenous) analyte displaced (or competed away) from a capture agent (e.g., an antibody specific for the analyte) by the analyte present in the sample. In one competitive assay, a known amount of, in this case, the protein of interest is added to the sample and the sample is then contacted with a capture agent, in this case an antibody that specifically binds to a polypeptide of the invention. The amount of immunogen bound to the antibody is inversely proportional to the concentration of immunogen present in the sample. In a particularly preferred embodiment, the antibody is immobilized on a solid substrate. For example, the amount of the polypeptide bound to the antibody may be determined either by measuring the amount of subject protein present in a protein/antibody complex or, alternatively, by measuring the amount of remaining uncomplexed protein. The amount of protein may be detected by providing a labeled protein molecule.

Immunoassays in the competitive binding format can be used for cross-reactivity determinations. For example, a protein of interest can be immobilized on a solid support. Proteins are added to the assay which compete with the binding of the antisera to the immobilized antigen. The ability of the above proteins to compete with the binding of the antisera to the immobilized protein is compared to that of the protein of interest. The percent cross-reactivity for the above proteins is calculated, using standard calculations. Those antisera with less than 10% cross-reactivity with each of the proteins listed above are selected and pooled. The cross-reacting antibodies are optionally removed from the pooled antisera by immunoabsorption with the considered proteins, e.g., distantly related homologs.

The immunoabsorbed and pooled antisera are then used in a competitive binding immunoassay as described above to compare a second protein, thought to be perhaps a protein of the present invention, to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein partially encoded by a sequence herein that is required, then the second protein is said to specifically bind to an antibody generated to an immunogen consisting of the target protein.

### 3. Other Assay Formats

In a particularly preferred embodiment, western blot (immunoblot) analysis is used to detect and quantify the presence of a polypeptide of the invention in the sample. The technique generally comprises separating sample proteins by gel electrophoresis on the basis of molecular weight, transferring the separated proteins to a suitable solid support (such as, e.g., a nitrocellulose filter, a nylon filter, or a derivatized nylon filter) and incubating the sample with the antibodies that specifically bind the protein of interest. For example, the antibodies specifically bind to a polypeptide of interest on the solid support. These antibodies may be directly labeled or alternatively may be subsequently detected using labeled antibodies (e.g., labeled sheep anti-mouse antibodies) that specifically bind to the antibodies against the protein of interest.

Other assay formats include liposome immunoassays (LIA), which use liposomes designed to bind specific molecules (e.g., antibodies) and release encapsulated reagents or markers. The released chemicals are then detected according to standard techniques (see, Monroe et al. (1986) Amer. Clin. Prod. Rev. 5:34-41).

### 4. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well developed in the field of immunoassays and, in general, most labels useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, the ease of conjugation with the compound, stability requirements, available instrumentation, and disposal provisions.

Non-radioactive labels are often attached by indirect means. The molecules can also be conjugated directly to signal generating compounds, e.g., by conjugation with an enzyme or fluorescent compound. A variety of enzymes and fluorescent compounds can be used with the methods of the present invention and are well-known to those of skill in the art (for a review of various labeling or signal producing systems which may be used, see, e.g., U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge-coupled devices (CCDs) or photomultipliers and the like. Similarly, enzymatic labels may be detected by providing the appropriate substrates for the enzyme and detecting the resulting reaction product. Finally simple colorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

In some embodiments, BP or MDD in a patient may be diagnosed or otherwise evaluated by visualizing expression in situ of one or more of the appropriately dysregulated gene sequences identified herein. Those skilled in the art of visualizing the presence or expression of molecules including nucleic acids, polypeptides and other biochemicals in the brains of living patients will appreciate that the gene expression information described herein may be utilized in the context of a variety of visualization methods. Such methods include, but are not limited to, single-photon emission-computed tomography (SPECT) and positron-emitting tomography (PET) methods. See, e.g., Vassaux and Groot-wassink, “In Vivo Noninvasive Imaging for Gene Therapy,” J. Biomedicine and Biotechnology, 2: 92-101 (2003).

PET and SPECT imaging shows the chemical functioning of organs and tissues, while other imaging techniques—such as X-ray, CT and MRI—show structure. The use of PET and SPECT imaging is useful for qualifying and monitoring the development of brain diseases, including schizophrenia and related disorders. In some instances, the use of PET or SPECT imaging allows diseases to be detected years earlier than the onset of symptoms. The use of small molecules for labelling and visualizing the presence or expression of polypeptides and nucleotides has had success, for example, in visualizing proteins in the brains of Alzheimer's patients, as described by, e.g., Herholz K et al., Mol Imaging Biol., 6(4):239-69 (2004); Nordberg A, Lancet Neurol., 3(9):519-27 (2004); Neuropsychol Rev., Zakzanis K K et al., 13(1):1-18 (2003); Kung M P et al, Brain Res., 1025(1-2):98-105 (2004); and Herholz K, Ann Nucl Med., 17(2):79-89 (2003).

The dysregulated exons disclosed in, e.g., Table 3, Table 4, and/or FIG. 1, or their encoded peptides (if any), or fragments thereof, can be used in the context of PET and SPECT imaging applications. After modification with appropriate tracer residues for PET or SPECT applications, molecules which interact or bind with any transcripts associated with the genes referenced in Table 3, Table 4, and/or FIG. 1, or with any polypeptides encoded by those transcripts may be used to visualize the patterns of gene expression and facilitate diagnosis of schizophrenia MDD or BP, as described herein. Similarly, if the encoded polypeptides encode enzymes, labeled molecules which interact with the products of catalysis by the enzyme may be used for the in vivo imaging and diagnostic application described herein.

Antisense technology is particularly suitable for detecting the transcripts identified in Table 3, Table 4, and/or FIG. 1 herein. For example, the use of antisense peptide nucleic acid (PNA) labeled with an appropriate radionuclide, such as ¹¹¹In, and conjugated to a brain drug-targeting system to enable transport across biologic membrane barriers, has been demonstrated to allow imaging of endogenous gene expression in brain cancer. See Suzuki et al., Journal of Nuclear Medicine, 10:1766-1775 (2004). Suzuki et al. utilize a delivery system comprising monoclonal antibodies that target transferring receptors at the blood-brain barrier and facilitate transport of the PNA across that barrier. Modified embodiments of this technique may be used to target any upregulated genes associated with schizophrenia, BP or MDD, such as any upregulated exons which appear in Table 3, Table 4, and/or FIG. 1, in methods of treating schizophrenic, BP or MDD patients.

In other embodiments, the dysregulated genes listed in Table 3, Table 4, and/or FIG. 1 may be used in the context of prenatal and neonatal diagnostic methods. For example, fetal or neonatal samples can be obtained and the expression levels of appropriate transcripts (e.g., the exon transcripts in Table 3, Table 4, and/or FIG. 1) may be measured and correlated with the presence or increased likelihood of a mental disorder, e.g., schizophrenia. Similarly, the presence of one or more of the SNPs identified in Table 1 or 2 may be used to infer or corroborate dysregulated expression of a gene and the likelihood of a mood disorder such as BP in prenatal, neonatal, children and adult patients.

In other embodiments, the brain labeling and imaging techniques described herein or variants thereof may be used in conjunction with any of the sequences in Tables 1-4 or FIG. 1 in a forensic analysis, i.e., to determine whether a deceased individual suffered from BP or schizophrenia.

### VI. Screening for Modulators of Polypeptides and Polynucleotides of the Invention

Modulators of polypeptides or polynucleotides of the invention, i.e. agonists or antagonists of their activity or modulators of polypeptide or polynucleotide expression, are useful for treating a number of human diseases, including mood disorders or psychotic disorders. Administration of agonists, antagonists or other agents that modulate expression of the polynucleotides or polypeptides of the invention can be used to treat patients with mood disorders or psychotic disorders.

### A. Screening Methods

A number of different screening protocols can be utilized to identify agents that modulate the level of expression or activity of polypeptides and polynucleotides of the invention in cells, particularly mammalian cells, and especially human cells. In general terms, the screening methods involve screening a plurality of agents to identify an agent that modulates the polypeptide activity by binding to a polypeptide of the invention, modulating inhibitor binding to the polypeptide or activating expression of the polypeptide or polynucleotide, for example.

### 1. Binding Assays

Preliminary screens can be conducted by screening for agents capable of binding to a polypeptide of the invention, as at least some of the agents so identified are likely modulators of polypeptide activity. The binding assays usually involve contacting a polypeptide of the invention with one or more test agents and allowing sufficient time for the protein and test agents to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet and Yamamura, (1985) “Neurotransmitter, Hormone or Drug Receptor Binding Methods,” in Neurotransmitter Receptor Binding (Yamamura, H. I., et al., eds.), pp. 61-89. The protein utilized in such assays can be naturally expressed, cloned or synthesized.

Binding assays are also useful, e.g., for identifying endogenous proteins that interact with a polypeptide of the invention. For example, antibodies, receptors or other molecules that bind a polypeptide of the invention can be identified in binding assays.

### 2. Expression Assays

Certain screening methods involve screening for a compound that up or down-regulates the expression of a polypeptide or polynucleotide of the invention. Such methods generally involve conducting cell-based assays in which test compounds are contacted with one or more cells expressing a polypeptide or polynucleotide of the invention and then detecting an increase or decrease in expression (either transcript, translation product, or catalytic product). Some assays are performed with peripheral cells, or other cells, that express an endogenous polypeptide or polynucleotide of the invention.

Polypeptide or polynucleotide expression can be detected in a number of different ways. As described infra, the expression level of a polynucleotide of the invention in a cell can be determined by probing the mRNA expressed in a cell with a probe that specifically hybridizes with a transcript (or complementary nucleic acid derived therefrom) of a polynucleotide of the invention. Probing can be conducted by lysing the cells and conducting Northern blots or without lysing the cells using in situ-hybridization techniques. Alternatively, a polypeptide of the invention can be detected using immunological methods in which a cell lysate is probed with antibodies that specifically bind to a polypeptide of the invention.

Other cell-based assays are reporter assays conducted with cells that do not express a polypeptide or polynucleotide of the invention. Certain of these assays are conducted with a heterologous nucleic acid construct that includes a promoter of a polynucleotide of the invention that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, chloramphenicol acetyl transferase (CAT); Alton and Vapnek (1979) Nature 282:864-869), luciferase, β-galactosidase, green fluorescent protein (GFP) and alkaline phosphatase (Toh, et al. (1980) Eur. J. Biochem. 182:231-238; and Hall et al. (1983) J. Mol. Appl. Gen. 2:101).

In these assays, cells harboring the reporter construct are contacted with a test compound. A test compound that either activates the promoter by binding to it or triggers a cascade that produces a molecule that activates the promoter causes expression of the detectable reporter. Certain other reporter assays are conducted with cells that harbor a heterologous construct that includes a transcriptional control element that activates expression of a polynucleotide of the invention and a reporter operably linked thereto. Here, too, an agent that binds to the transcriptional control element to activate expression of the reporter or that triggers the formation of an agent that binds to the transcriptional control element to activate reporter expression, can be identified by the generation of signal associated with reporter expression.

The level of expression or activity can be compared to a baseline value. As indicated above, the baseline value can be a value for a control sample or a statistical value that is representative of expression levels for a control population (e.g., healthy individuals not having or at risk for mood disorders or psychotic disorders). Expression levels can also be determined for cells that do not express a polynucleotide of the invention as a negative control. Such cells generally are otherwise substantially genetically the same as the test cells.

A variety of different types of cells can be utilized in the reporter assays. Cells that express an endogenous polypeptide or polynucleotide of the invention include, e.g., brain cells, including cells from the cerebellum, anterior cingulate cortex, dorsolateral prefrontal cortex, amygdala, hippocampus, or nucleus accumbens. Cells that do not endogenously express polynucleotides of the invention can be prokaryotic, but are preferably eukaryotic. The eukaryotic cells can be any of the cells typically utilized in generating cells that harbor recombinant nucleic acid constructs. Exemplary eukaryotic cells include, but are not limited to, yeast, and various higher eukaryotic cells such as the COS, CHO and HeLa cell lines.

Various controls can be conducted to ensure that an observed activity is authentic including running parallel reactions with cells that lack the reporter construct or by not contacting a cell harboring the reporter construct with test compound. Compounds can also be further validated as described below.

### 3. Catalytic Activity

Catalytic activity of polypeptides of the invention can be determined by measuring the production of enzymatic products or by measuring the consumption of substrates. Activity refers to either the rate of catalysis or the ability to the polypeptide to bind (Kₘ) the substrate or release the catalytic product (K_{d}).

Analysis of the activity of polypeptides of the invention are performed according to general biochemical analyses. Such assays include cell-based assays as well as in vitro assays involving purified or partially purified polypeptides or crude cell lysates. The assays generally involve providing a known quantity of substrate and quantifying product as a function of time.

### 4. Validation

Agents that are initially identified by any of the foregoing screening methods can be further tested to validate the apparent activity. Preferably such studies are conducted with suiTable 1nimal models. The basic format of such methods involves administering a lead compound identified during an initial screen to an animal that serves as a model for humans and then determining if expression or activity of a polynucleotide or polypeptide of the invention is in fact upregulated. The animal models utilized in validation studies generally are mammals of any kind. Specific examples of suiTable 1nimals include, but are not limited to, primates, mice, and rats. As described herein, models using administration of known therapeutics can be useful.

### 5. Animal Models

Animal models of mental disorders also find use in screening for modulators. In one embodiment, invertebrate models such as Drosophila models can be used, screening for modulators of Drosophila orthologs of the human genes disclosed herein. In another embodiment, transgenic animal technology including gene knockout technology, for example as a result of homologous recombination with an appropriate gene targeting vector, or gene overexpression, will result in the absence, decreased or increased expression of a polynucleotide or polypeptide of the invention. The same technology can also be applied to make knockout cells. When desired, tissue-specific expression or knockout of a polynucleotide or polypeptide of the invention may be necessary. Transgenic animals generated by such methods find use as animal models of mental illness and are useful in screening for modulators of mental illness.

Knockout cells and transgenic mice can be made by insertion of a marker gene or other heterologous gene into an endogenous gene site in the mouse genome via homologous recombination. Such mice can also be made by substituting an endogenous polynucleotide of the invention with a mutated version of the polynucleotide, or by mutating an endogenous polynucleotide, e.g., by exposure to carcinogens.

For development of appropriate stem cells, a DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

### B. Modulators of Polypeptides or Polynucleotides of the Invention

The agents tested as modulators of the polypeptides or polynucleotides of the invention can be any small chemical compound, or a biological entity, such as a protein, sugar, nucleic acid or lipid. Alternatively, modulators can be genetically altered versions of a polypeptide or polynucleotide of the invention. Typically, test compounds will be small chemical molecules and peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like. Modulators also include agents designed to reduce the level of mRNA of the invention (e.g. antisense molecules, ribozymes, DNAzymes and the like) or the level of translation from an mRNA.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al, Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences, Columbia, Md., etc.).

### C. Solid State and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds are possible using the integrated systems of the invention. More recently, microfluidic approaches to reagent manipulation have been developed.

The molecule of interest can be bound to the solid state component, directly or indirectly, via covalent or non-covalent linkage, e.g., via a tag. The tag can be any of a variety of components. In general, a molecule that binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs, such as agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-Gly sequences of between about 5 and 200 amino acids. Such flexible linkers are known to those of skill in the art. For example, poly(ethelyne glycol) linkers are available from Shearwater Polymers, Inc., Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (see, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank and Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

The invention provides in vitro assays for identifying, in a high throughput format, compounds that can modulate the expression or activity of the polynucleotides or polypeptides of the invention. In a preferred embodiment, the methods of the invention include such a control reaction. For each of the assay formats described, “no modulator” control reactions that do not include a modulator provide a background level of binding activity.

In some assays it will be desirable to have positive controls to ensure that the components of the assays are working properly. At least two types of positive controls are appropriate. First, a known activator of a polynucleotide or polypeptide of the invention can be incubated with one sample of the assay, and the resulting increase in signal resulting from an increased expression level or activity of polynucleotide or polypeptide determined according to the methods herein. Second, a known inhibitor of a polynucleotide or polypeptide of the invention can be added, and the resulting decrease in signal for the expression or activity can be similarly detected.

### D. Computer-Based Assays

Yet another assay for compounds that modulate the activity of a polypeptide or polynucleotide of the invention involves computer assisted drug design, in which a computer system is used to generate a three-dimensional structure of the polypeptide or polynucleotide based on the structural information encoded by its amino acid or nucleotide sequence. The input sequence interacts directly and actively with a pre-established algorithm in a computer program to yield secondary, tertiary, and quaternary structural models of the molecule. Similar analyses can be performed on potential receptors or binding partners of the polypeptides or polynucleotides of the invention. The models of the protein or nucleotide structure are then examined to identify regions of the structure that have the ability to bind, e.g., a polypeptide or polynucleotide of the invention. These regions are then used to identify polypeptides that bind to a polypeptide or polynucleotide of the invention.

The three-dimensional structural model of a protein is generated by entering protein amino acid sequences of at least 10 amino acid residues or corresponding nucleic acid sequences encoding a potential receptor into the computer system. The amino acid sequences encoded by the nucleic acid sequences provided herein represent the primary sequences or subsequences of the proteins, which encode the structural information of the proteins. At least 10 residues of an amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary, and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of a polypeptide or polynucleotide of the invention to identify binding sites of the polypeptide or polynucleotide of the invention. Binding affinity between the protein and ligands is determined using energy terms to determine which ligands have an enhanced probability of binding to the protein.

Computer systems are also used to screen for mutations, polymorphic variants, alleles and interspecies homologs of genes encoding a polypeptide or polynucleotide of the invention. Such mutations can be associated with disease states or genetic traits and can be used for diagnosis. As described above, GeneChip™ and related technology can also be used to screen for mutations, polymorphic variants, alleles and interspecies homologs. Once the variants are identified, diagnostic assays can be used to identify patients having such mutated genes. Identification of the mutated a polypeptide or polynucleotide of the invention involves receiving input of a first amino acid sequence of a polypeptide of the invention (or of a first nucleic acid sequence encoding a polypeptide of the invention), e.g., any amino acid sequence having at least 60%, optionally at least 70% or 85%, identity with the amino acid sequence of interest, or conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various polynucleotides of the invention, and mutations associated with disease states and genetic traits.

### VII. Compositions, Kits and Integrated Systems

The invention provides compositions, kits and integrated systems for practicing the assays described herein using polypeptides or polynucleotides of the invention, antibodies specific for polypeptides or polynucleotides of the invention, etc.

The invention provides assay compositions for use in solid phase assays; such compositions can include, for example, one or more polynucleotides or polypeptides of the invention immobilized on a solid support, and a labeling reagent. In each case, the assay compositions can also include additional reagents that are desirable for hybridization. Modulators of expression or activity of polynucleotides or polypeptides of the invention can also be included in the assay compositions.

The invention also provides kits for carrying out the therapeutic and diagnostic assays of the invention. The kits typically include a probe that comprises an antibody that specifically binds to polypeptides or polynucleotides of the invention, and a label for detecting the presence of the probe. The kits may include several polynucleotide sequences encoding polypeptides of the invention. Kits can include any of the compositions noted above, and optionally further include additional components such as instructions to practice a high-throughput method of assaying for an effect on expression of the genes encoding the polypeptides of the invention, or on activity of the polypeptides of the invention, one or more containers or compartments (e.g., to hold the probe, labels, or the like), a control modulator of the expression or activity of polypeptides of the invention, a robotic armature for mixing kit components or the like.

The invention also provides integrated systems for high-throughput screening of potential modulators for an effect on the expression or activity of the polypeptides of the invention. The systems typically include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous STAT binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS®, OS2® WINDOWS®, WINDOWS NT®, WINDOWS95®, WINDOWS98®, or WINDOWS2000® based computers), MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

### VIII. Administration and Pharmaceutical Compositions

Modulators of the polynucleotides or polypeptides of the invention (e.g., antagonists or agonists) can be administered directly to a mammalian subject for modulation of activity of those molecules in vivo. Administration is by any of the routes normally used for introducing a modulator compound into ultimate contact with the tissue to be treated and is well known to those of skill in the art. Although more than one route can be used to administer a particular composition, a particular route can often provide a more immediate and more effective reaction than another route.

Diseases that can be treated include the following, which include the corresponding reference number from Morrison, DSM-IV Made Easy, 1995: Schizophrenia, Catatonic, Subchronic, (295.21); Schizophrenia, Catatonic, Chronic (295.22); Schizophrenia, Catatonic, Subchronic with Acute Exacerbation (295.23); Schizophrenia, Catatonic, Chronic with Acute Exacerbation (295.24); Schizophrenia, Catatonic, in Remission (295.55); Schizophrenia, Catatonic, Unspecified (295.20); Schizophrenia, Disorganized, Subchronic (295.11); Schizophrenia, Disorganized, Chronic (295.12); Schizophrenia, Disorganized, Subchronic with Acute Exacerbation (295.13); Schizophrenia, Disorganized, Chronic with Acute Exacerbation (295.14); Schizophrenia, Disorganized, in Remission (295.15); Schizophrenia, Disorganized, Unspecified (295.10); Schizophrenia, Paranoid, Subchronic (295.31); Schizophrenia, Paranoid, Chronic (295.32); Schizophrenia, Paranoid, Subchronic with Acute Exacerbation (295.33); Schizophrenia, Paranoid, Chronic with Acute Exacerbation (295.34); Schizophrenia, Paranoid, in Remission (295.35); Schizophrenia, Paranoid, Unspecified (295.30); Schizophrenia, Undifferentiated, Subchronic (295.91); Schizophrenia, Undifferentiated, Chronic (295.92); Schizophrenia, Undifferentiated, Subchronic with Acute Exacerbation (295.93); Schizophrenia, Undifferentiated, Chronic with Acute Exacerbation (295.94); Schizophrenia, Undifferentiated, in Remission (295.95); Schizophrenia, Undifferentiated, Unspecified (295.90); Schizophrenia, Residual, Subchronic (295.61); Schizophrenia, Residual, Chronic (295.62); Schizophrenia, Residual, Subchronic with Acute Exacerbation (295.63); Schizophrenia, Residual, Chronic with Acute Exacerbation (295.94); Schizophrenia, Residual, in Remission (295.65); Schizophrenia, Residual, Unspecified (295.60); Delusional (Paranoid) Disorder (297.10); Brief Reactive Psychosis (298.80); Schizophreniform Disorder (295.40); Schizoaffective Disorder (295.70); Induced Psychotic Disorder (297.30); Psychotic Disorder NOS (Atypical Psychosis) (298.90); Personality Disorders, Paranoid (301.00); Personality Disorders, Schizoid (301.20); Personality Disorders, Schizotypal (301.22); Personality Disorders, Antisocial (301.70); Personality Disorders, Borderline (301.83) and bipolar disorders, maniac, hypomaniac, dysthymic or cyclothymic disorders, substance-induced mood disorders, major depression, psychosis, including paranoid psychosis, catatonic psychosis, delusional psychosis, having schizoaffective disorder, and substance-induced psychotic disorder.

In some embodiments, modulators of polynucleotides or polypeptides of the invention can be combined with other drugs useful for treating mental disorders including useful for treating mood disorders, e.g., schizophrenia, bipolar disorders, or major depression. In some preferred embodiments, pharmaceutical compositions of the invention comprise a modulator of a polypeptide of polynucleotide of the invention combined with at least one of the compounds useful for treating schizophrenia, bipolar disorder, or major depression, e.g., such as those described in U.S. Pat. Nos. 6,297,262; 6,284,760; 6,284,771; 6,232,326; 6,187,752; 6,117,890; 6,239,162 or 6,166,008.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)).

The modulators (e.g., agonists or antagonists) of the expression or activity of the a polypeptide or polynucleotide of the invention, alone or in combination with other suitable components, can be made into aerosol formulations (i.e., they can be “nebulized”) to be administered via inhalation or in compositions useful for injection. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to effect a beneficial response in the subject over time. The optimal dose level for any patient will depend on a variety of factors including the efficacy of the specific modulator employed, the age, body weight, physical activity, and diet of the patient, on a possible combination with other drugs, and on the severity of the mental disorder. The size of the dose also will be determined by the existence, nature, and extent of any adverse side effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side effects of the modulator at various concentrations, as applied to the mass and overall health of the subject. Administration can be accomplished via single or divided doses.

### IX. Gene Therapy Applications

A variety of human diseases can be treated by therapeutic approaches that involve stably introducing a gene into a human cell such that the gene is transcribed and the gene product is produced in the cell. Diseases amenable to treatment by this approach include inherited diseases, including those in which the defect is in a single or multiple genes. Gene therapy is also useful for treatment of acquired diseases and other conditions. For discussions on the application of gene therapy towards the treatment of genetic as well as acquired diseases, see, Miller, Nature 357:455-460 (1992); and Mulligan, Science 260:926-932 (1993).

In the context of the present invention, gene therapy can be used for treating a variety of disorders and/or diseases in which the polynucleotides and polypeptides of the invention has been implicated. For example, compounds, including polynucleotides, can be identified by the methods of the present invention as effective in treating a mental disorder. Introduction by gene therapy of these polynucleotides can then be used to treat, e.g., mental disorders including mood disorders and psychotic disorders.

### A. Vectors for Gene Delivery

For delivery to a cell or organism, the polynucleotides of the invention can be incorporated into a vector. Examples of vectors used for such purposes include expression plasmids capable of directing the expression of the nucleic acids in the target cell. In other instances, the vector is a viral vector system wherein the nucleic acids are incorporated into a viral genome that is capable of transfecting the target cell. In a preferred embodiment, the polynucleotides can be operably linked to expression and control sequences that can direct expression of the gene in the desired target host cells. Thus, one can achieve expression of the nucleic acid under appropriate conditions in the target cell.

### B. Gene Delivery Systems

Viral vector systems useful in the expression of the nucleic acids include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the genes of interest are inserted into such vectors to allow packaging of the gene construct, typically with accompanying viral DNA, followed by infection of a sensitive host cell and expression of the gene of interest.

As used herein, “gene delivery system” refers to any means for the delivery of a nucleic acid of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221, WO 93/14188, and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, molecular conjugates of the instant invention can include microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors are also useful for introducing the nucleic acids of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis-acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide or polynucleotide of the invention and thus restore the cells to a normal phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

In some embodiments of the invention, an antisense polynucleotide is administered which hybridizes to a gene encoding a polypeptide of the invention. The antisense polypeptide can be provided as an antisense oligonucleotide (see, e.g., Murayama et al., Antisense Nucleic Acid Drug Dev. 7:109-114 (1997)). Genes encoding an antisense nucleic acid can also be provided; such genes can be introduced into cells by methods known to those of skill in the art. For example, one can introduce an antisense nucleotide sequence in a viral vector, such as, for example, in hepatitis B virus (see, e.g., Ji et al., J. Viral Hepat. 4:167-173 (1997)), in adeno-associated virus (see, e.g., Xiao et al., Brain Res. 756:76-83 (1997)), or in other systems including, but not limited, to an HVJ (Sendai virus)-liposome gene delivery system (see, e.g., Kaneda et al., Ann. NY Acad. Sci. 811:299-308 (1997)), a “peptide vector” (see, e.g., Vidal et al., CR Acad. Sci III 32:279-287 (1997)), as a gene in an episomal or plasmid vector (see, e.g., Cooper et al., Proc. Natl. Acad. Sci. U.S.A. 94:6450-6455 (1997), Yew et al. Hum Gene Ther. 8:575-584 (1997)), as a gene in a peptide-DNA aggregate (see, e.g., Niidome et al., J. Biol. Chem. 272:15307-15312 (1997)), as “naked DNA” (see, e.g., U.S. Pat. Nos. 5,580,859 and 5,589,466), in lipidic vector systems (see, e.g., Lee et al., Crit Rev Ther Drug Carrier Syst. 14:173-206 (1997)), polymer coated liposomes (U.S. Pat. Nos. 5,213,804 and 5,013,556), cationic liposomes (Epand et al., U.S. Pat. Nos. 5,283,185; 5,578,475; 5,279,833; and 5,334,761), gas filled microspheres (U.S. Pat. No. 5,542,935), ligand-targeted encapsulated macromolecules (U.S. Pat. Nos. 5,108,921; 5,521,291; 5,554,386; and 5,166,320).

Upregulated transcripts listed in the biomarker tables herein which are correlated with mental disorders may be targeted with one or more short interfering RNA (siRNA) sequences that hybridize to specific sequences in the target, as described above. Targeting of certain brain transcripts with siRNA in vivo has been reported, for example, by Zhang et al., J. Gene. Med., 12:1039-45 (2003), who utilized monoclonal antibodies against the transferrin receptor to facilitate passage of liposome-encapsulated siRNA molecules through the blood brain barrier. Targeted siRNAs represent useful therapeutic compounds for attenuating the over-expressed transcripts that are associated with disease states, e.g., MDD, BP, and other mental disorders.

In another embodiment, conditional expression systems, such as those typified by the tet-regulated systems and the RU-486 system, can be used (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These systems impart small molecule control on the expression of the target gene(s) of interest.

In another embodiment, stem cells engineered to express a transcript of interest can implanted into the brain.

### C. Pharmaceutical Formulations

When used for pharmaceutical purposes, the vectors used for gene therapy are formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer, or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans; antioxidants, such as ascorbic acid or glutathione; chelating agents; low molecular weight proteins or other stabilizers or excipients. Other physiologically acceptable compounds include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers, or adjuvants can be found in Remington's Pharmaceutical Sciences, Mack Publishing Company, Philadelphia, Pa., 17th ed. (1985).

### D. Administration of Formulations

The formulations of the invention can be delivered to any tissue or organ using any delivery method known to the ordinarily skilled artisan. In some embodiments of the invention, the nucleic acids of the invention are formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701.

### E. Methods of Treatment

The gene therapy formulations of the invention are typically administered to a cell. The cell can be provided as part of a tissue, such as an epithelial membrane, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced into cells by such methods as microinjection, calcium phosphate precipitation, liposome fusion, or biolistics. In further embodiments, the nucleic acids are taken up directly by the tissue of interest.

In some embodiments of the invention, the nucleic acids of the invention are administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23 (1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

### X. Diagnosis of Mood Disorders and Psychotic Disorders

The present invention also provides methods of diagnosing mood disorders (such as major depression or bipolar disorder), psychotic disorders (such as schizophrenia), or a predisposition of at least some of the pathologies of such disorders. Diagnosis may involve determining the level of a polypeptide or polynucleotide of the invention in a patient and then comparing the level to a baseline or range. Typically, the baseline value is representative of a polypeptide or polynucleotide of the invention in a healthy person not suffering from a mood disorder or a psychotic disorder or under the effects of medication or other drugs. Variation of levels of a polypeptide or polynucleotide of the invention from the baseline range (either up or down) indicates that the patient has a mood disorder or a psychotic disorder or at risk of developing at least some aspects of a mood disorder or a psychotic disorder. In some embodiments, the level of a polypeptide or polynucleotide of the invention are measured by taking a blood, urine or tissue sample from a patient and measuring the amount of a polypeptide or polynucleotide of the invention in the sample using any number of detection methods, such as those discussed herein.

Antibodies can be used in assays to detect differential protein expression in patient samples, e.g., ELISA assays, immunoprecipitation assays, and immunohistochemical assays. PCR assays can be used to detect expression levels of nucleic acids, as well as to measure levels of transcription of particular exons (e.g., in DSC2).

In the case where absence of gene expression is associated with a disorder, the genomic structure of a gene can be evaluated with known methods such as PCR to detect deletion or insertion mutations associated with disease susceptibility. Conversely, the presence of mRNA or protein corresponding to the gene would indicate that an individual does not have susceptibility to BP. Thus, diagnosis can be made by detecting the presence or absence of mRNA or protein, or by examining the genomic structure of the gene, e.g., by detecting the presence or absence of an SNP such as the SNPs listed in Tables 1 and 2.

Single nucleotide polymorphism (SNP) analysis is useful for detecting differences between alleles of the polynucleotides (e.g., genes) of the invention. SNPs such as those listed in Tables 1 and 2 are useful, for instance, for diagnosis of diseases (e.g., bipolar disorder) whose occurrence is linked to the gene sequences of the invention. For example, if an individual carries at least one SNP linked to a BP-associated allele of the gene sequences of the invention, the individual is likely predisposed for BP. If the individual is homozygous for a disease-linked SNP, the individual is particularly predisposed for occurrence of that disease. In some embodiments, the SNP associated with the gene sequences of the invention is located within 300,000; 200,000; 100,000; 75,000; 50,000; or 10,000 base pairs from the gene sequence.

Various real-time PCR methods can be used to detect the SNPs of Table 1 and 2, including, e.g., Taqman or molecular beacon-based assays (e.g., U.S. Pat. Nos. 5,210,015; 5,487,972; Tyagi et al., Nature Biotechnology 14:303 (1996); and PCT WO 95/13399 are useful to monitor for the presence of absence of a SNP. Additional SNP detection methods include, e.g., DNA sequencing, sequencing by hybridization, dot blotting, oligonucleotide array (DNA Chip) hybridization analysis, or are described in, e.g., U.S. Pat. No. 6,177,249; Landegren et al., Genome Research, 8:769-776 (1998); Botstein et al., Am J Human Genetics 32:314-331 (1980); Meyers et al., Methods in Enzymology 155:501-527 (1987); Keen et al., Trends in Genetics 7:5 (1991); Myers et al., Science 230:1242-1246 (1985); and Kwok et al., Genomics 23:138-144 (1994). PCR methods can also be used to detect deletion/insertion polymorphisms.

In some embodiments, the level of the enzymatic product of a polypeptide or polynucleotide of the invention is measured and compared to a baseline value of a healthy person or persons. Modulated levels of the product compared to the baseline indicates that the patient has a mood disorder or a psychotic disorder or is at risk of developing at least some aspects of a mood disorder or a psychotic disorder. Patient samples, for example, can be blood, urine or tissue samples. The genes disclosed herein may be used as biomarkers for detecting and treating BP and schizophrenia.

The invention also provides nucleic acid sequences and protein sequences which are useful for deciphering the mode of action of currently used mood stabilizers such as lithium. The sequences provided are also useful for drug discovery, e.g., discovering new leads to identifying more efficacious therapeutic targets in the form of a central molecule/pathway through which an entire system or network of pathways can be modulated to remedy the perturbed cellular process underlying schizophrenia, BP, or a principal endophenotype of these disorders. Improved knowledge of target-specificity of drugs could help to minimize side effects associated with numerous mood stabilizers currently in use. It could also facilitate development of a subset of biomarker genes useful in early diagnosis of BP or schizophrenia, and in monitoring drug efficacy.

### XI. Determination of Linkage Disequilibrium

LD is the non-random association of alleles adjacent loci. When a particular allele at one locus is found together on the same chromosome with a specific allele at a second locus—more often than expected if the loci were segregating independently in a population—the loci are in disequilibrium. This concept of LD is formalized by one of the earliest measures of disequilibrium to be proposed (symbolized by D) (Lewontin, R. C.; Genetics (1964) 49, 49-67). D, in common with most other measures of LD, quantifies disequilibrium as the difference between the observed frequency of a two-locus haplotype and the frequency it would be expected to show if the alleles are segregating at random. Adopting the standard notation for two adjacent loci—A and B, with two alleles (A, a and B, b) at each locus—the observed frequency of the haplotype that consists of alleles A and B is represented by P_{AB}. Assuming the independent assortment of alleles at the two loci, the expected halotype frequency is calculated as the product of the allele frequency of each of the two alleles, or P_{A}×P_{B}, where P_{A} is the frequency of allele A at the first locus and P_{B} is the frequency of allele B at the second locus. So, one of the simplest measures of disequilibrium is

D=P_{AB}−P_{A}×P_{B}

LD is created when a new mutation occurs on a chromosome that carries a particular allele at a nearby locus, and is gradually eroded by recombination. Recurrent mutations can also lessen the association between alleles at adjacent loci.

The importance of recombination in shaping patterns of LD is acknowledged by the moniker of “linkage”. The extent of LD in populations is expected to decrease with both time (t) and recombinational distance (r, or the recombination fraction) between markers. Theoretically, LD decays with time and distance according to the following formula, where D₀ is the extent of disequilibrium at some starting point and Dₜ, is the extent of disequilibrium t generation later:

Dₜ=(1−r)^{t}D₀

Although the measure D has the intuitive concepts of LD, its numerical value is of little use for measuring the strength of and comparing levels of LD. This is due to the dependence of D on allele frequencies. The two most common measures are the absolute value of D′ and r².

The absolute value of D′ is determined by dividing D by its maximum possible value, given the allele frequencies at the two loci. The case of D′=1 is known as “complete LD”. Values of D′<1 indicate that the complete ancestral LD has been disrupted. The magnitude of values of D′<1 has no clear interpretation. Estimates of D′ are strongly inflated in small samples. Therefore, statistically significant values of D′ that are near one provide a useful indication of minimal historical recombination, but intermediate values should not be used for comparisons of the strength of LD between studies, or to measure the extent of LD.

The measure r² is in some ways complementary to D′. r² is equal to D² divided by the product of the allele frequencies at the two loci. Hill and Roberson deduced that E [r²]=1/1+4Nc where c is the recombination rate in morgans between the two markers and N is the effective population size. This equation illustrates two important properties of LD. First, expected levels of LD are a function of recombination. The more recombination between two sites, the more they are shuffled with respect to one another, decreasing LD. Second, LD is a function of N, emphasizing that LD is a property of populations. To arrive at this equation, Hill and Roberson (Theor. Appl. Genet. (1968) 226-231) assumed that the population was an “ideal” large, random-mating population without natural selection and mutation.

Variants for BP1 risk thus include those in LD (likely r²>0.3 or D′>0.75) with the SNPs listed in Table 1 and/or Table 2. Genes or genomic elements affected by any causative SNP are most likely within 100 kb, but could be further away.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLE 1

### Whole Genome Study to Identify SNPs Associated with BP Disease

This example compares the genotype frequencies of BPI individuals to control individuals with no reported BPI, schizophrenia or major depression.

Sample selection: 1,160 Bipolar I (BPI) cases were selected from Distribution 3.07 of the NIMH Human Genetics Initiative repository and 57 Bipolar I cases from the Heinz Prechter repository of the University of Michigan Depression Center. The NIMH cases were diagnosed with BPI and came from 10 study sites within the United States. Of available families, we initially selected one BPI individual with ethnicity reported as described below. When available, we also selected a second BPI sibling. Sibships containing the proband were preferentially selected. In total, 489 sibpairs and 182 singleton BPI cases were selected. Subjects from the Prechter repository were either self-referred (by an advertisement on the depression center's Website) or were recruited during a clinical visit. Subects in both the NIMH and Prechter repository were administered the Diagnostic Interview for Genetic Studies (DIGS) and the consensus of two clinicians was used to diagnose BPI.

We selected 792 controls from Distribution 5 of the NIMH repository (individuals participating in an internet survey). Controls were white subjects aged 20 to 70. Individuals that reported having schizophrenia, bipolar disorder, or having heard voices that others could not hear were excluded. Individuals with suspected major depression were also excluded based on the responses to the psychiatric screening interview. From these individuals, controls were selected with grandparental ethnic backgrounds matched to the cases.

Ethnicity matching: We matched cases and controls for reported ethnicity based on the ethnic backgrounds recorded using information from the Diagnostic Interview for Genetic Studies (DIGS). Sixteen ethnicity categories are defined in the DIGS. The NIMH case subjects could report up to four ethnicities for each of their parents, and the NIMH control subjects could report up to sixteen ethnicities for each of their grandparents. Prechter cases could report a vector of 16 ethnicity variables (race 1-16) that was generated for each individual. Each ethnicity variable was assigned the sum of the parental or grandparental reports. The ethnicity variables were normalized so that the sum of the 16 variables equaled 1. To simply the matching combined the Anglo-Saxon, Northern Europe, and Western Europe categories (North/West Europe) into one category and the Russian and Eastern Europe categories (Russian/Eastern Europe) into a second category. For NIMH cases we restricted our initial control matching to the singleton case or a BPI sibling that only reported membership in European ancestry categories (Anglo-Saxon, Northern Europe, Eastern Europe/Slavic, Western Europe, Russian, and Mediterranean). If possible, a control was selected with the same proportion of ancestry in the North/West Europe, Russian/Eastern Europe and Mediterranean variables. When no exact match was possible, the European ancestry-only control with the minimum summed square difference in the ethnicity vectors was selected. Next the controls (no restriction on ethnicity) were matched preferential to BPI siblings that did not report only European ancestry and then to other second siblings of an original matched sibling. Fifty-seven controls were matched to the Prechter cases.

Quality control measures: Genotyping was performed in two rounds. The first round at the University of Michigan and Stanford University Genome Center (466 cases and 426 controls) and the second round at Stanford University Genome Center (747 cases and 326 controls). Samples from the two rounds were clustered separately based on the genotype data using cluster boundaries determined with our own data.

We checked for consistency in genotyping within duplicate sample pairs and with Hardy-Weinberg Equilibrium (HWE) using the unrelated individuals. We calculated the identity by state relationship between all of the samples using PLINK to verify the expected relationships between samples.

SNPs were dropped from all analyses if the HWE p-value was <10-6, the total number of duplicate pair discrepancies was >2 in either phase, the SNP call rate was <95% in either phase or overall the minor allele frequency <1%. 514,722 autosomal SNPs met our quality control criteria. All genotypes were oriented to the forward strand. There is little risk of strand ambiguities as there are no C/G or A/T polymorphisms included in the Illumina 300K HumanHap panel.

For the 519,223 autosomal SNPs (before quality control exclusions) with minor allele frequency >1% the genotype consistency rate among duplicate sample pairs was 99.997% for phase 1 and 99.984% for phase 2.

Statistical analysis: To empirically assess the degree of population stratification and the possibility of residual imbalance between cases and controls, we jointly analyzed our samples and a reference set consisting of 156 European samples that are part of the Human Genome Diversity Project (HGDP) panel. These samples represent eight European populations (17 Adygei, 24 Basque, 28 French, 12 Northern Italian, 15 Orcadian, 25 Russian, 28 Sardinian, and 7 Tuscan individuals) and have been genotyped in a separate study on Illumina HumanHap650 Beadchips. We first analyzed the 156 reference samples and identified top 20,000 most informative markers for characterizing within-Europe genetic diversity. Of these, ˜17,300 overlap with the HumanHap550chips (which we used in the study of bipolar disorder). We performed a principal component analysis of the 156 samples at these 17,300 loci, and observed that the first two components adequately separate the eight reference populations. We then used the first two eigenvectors (the “loadings” for the first two principal components) and the 17,300-SNP genotype data for bipolar study samples to calculate each sample's principal component scores along the first two components. This analysis tried to project the European American samples along the main axes of genetic variation defined by the reference European samples. Most of our Anglo-Saxon, Northern European, and Western European samples are indeed of northem/western European origin. None of our samples appears to have a significant non-European ancestry. The Eastern European/Slavic samples show a moderate proximity to the reference Russian samples, whereas the Russian and Mediterranean samples are mostly similar to the reference Italian samples, suggesting a southern Europe origin. The 3rd to sixth components among the 156 samples are also “meaningful”, as they are driven by the Basque, Italian, Russian, and French samples, respectively (not shown). The PC scores of our samples along each of the six axes show good balance between the cases and controls, p-value ˜0.2-0.9, for t-tests comparing the case and control PC scores for each of the six axes.

We performed a case-control association analysis using a standard chi-square statistic with the variance of the test statistic corrected for the relationship between the affected siblings (Bourgain et al. 2003). This method tests for an allele frequency difference between cases and controls assuming an additive model. We estimated the genomic control value and found a lambda of approximately 1 suggesting little evidence of population stratification.

DNA Handling Protocols: DNA was purchased from Rutgers. The genomic DNA processing and amplification were performed as defined for the Ilumina Infinium II genotyping platform. Briefly, 750 ng of human gDNA is isothermally amplified overnight. The amplified DNA is then fragmented by controlled enzymatic digestion. The DNA is then concentrated by precipitation and hybridized to Illumina Infinium II arrays. Amplified and fragmented DNA samples hybridize to locus specific 50-mers (on beads). Each bead type (>500,000 bed types) corresponds to each allele per SNP locus. Following hybridization, allelic specificity is conferred by enzymatic base extension and revealed by fluorescent staining.

Additional analysis: The core gene-set analysis algorithms were adopted to GWA data analysis by adding (1) support for SNP genotype data and SNP statistics such as Chi square test; (2) weighting mechanisms for correcting the dependence of multiple SNPs in the same linkage disequilibrium region within the same SNP group; and (3) SNP function group definitions using existing knowledge, such as Entrez Gene, Gene Ontology, KEGG/BioCarta/GenMAPP pathways, cytobands, differentially expressed genes from microarray study, potential targets of microRNA, etc.

The top 44 SNPs associated with Bipolar disorder are shown in Table 1, below. The p_SNP p-value represents the minimum p-value from the dominant, recessive and multiplicate tests, corrected for the performance of the above-mentioned three tests.

TABLE 1

| | | | p_SNP | |
|---|---|---|---|---|
| SNP | chr | pos | p-value | Other SNPs in this bin |
| rs6661361 | 1 | 195025682 | 0.00001093 | rs12059603; rs4915269; rs10922418; rs4412625; |
| | | | | rs4525073; rs2813164; rs10737570 |
| rs10737570 | 1 | 185492285 | 0.00009468 | |
| rs940052 | 2 | 45892793 | 4.24E−06 | rs2528614; rs1533476 |
| rs2528614 | 2 | 159389783 | 0.00001736 | rs757926; rs925781; rs925781; rs1990153 |
| rs4443010 | 2 | 111185369 | 0.0000507 | |
| rs13392378 | 2 | 28518995 | 0.0000685 | |
| rs1553092 | 3 | 189225286 | 0.00002016 | |
| rs10511422 | 3 | 125183858 | 0.00005062 | |
| rs7658020 | 4 | 96934988 | 0.00008401 | |
| rs7676537 | 4 | 109221339 | 0.00009034 | |
| rs743682 | 4 | 1765083 | 0.0000948 | |
| rs4691753 | 4 | 162675591 | 0.00009858 | |
| rs6882857 | 5 | 108960779 | 6.87E−07 | rs4957576; rs1490776; rs902505 |
| rs1045706 | 5 | 108742197 | 0.00001328 | rs1862205; rs400277; rs7705657 |
| rs1490996 | 5 | 124974431 | 0.00007175 | |
| rs9368392 | 6 | 22128881 | 0.00005161 | |
| rs1529015 | 6 | 147389383 | 0.00005464 | |
| rs9353722 | 6 | 91162721 | 0.00005795 | |
| rs4960221 | 6 | 6547432 | 0.00007431 | |
| rs9648517 | 7 | 41804347 | 0.00002912 | |
| rs1118380 | 7 | 51774097 | 0.00004132 | rs452247 |
| rs12056107 | 7 | 137349503 | 0.00007844 | rs6467744 |
| rs4907399 | 8 | 142624890 | 4.48E−06 | |
| rs10505292 | 8 | 118102508 | 0.00008076 | |
| rs2905072 | 9 | 132874589 | 0.00003808 | |
| rs10989791 | 9 | 101897154 | 0.00008514 | |
| rs11141719 | 9 | 87084040 | 0.00008935 | |
| rs3750895 | 10 | 101107547 | 0.00002532 | |
| rs4409766 | 10 | 104606653 | 0.00003556 | rs3824754; rs12411886; rs11191425; |
| | | | | rs12413409 |
| rs9423466 | 10 | 3152669 | 0.00004039 | rs7086721 |
| rs10881732 | 10 | 91772708 | 0.00006577 | rs4933526 |
| rs174537 | 11 | 61309256 | 8.11E−06 | rs174611; rs174576; rs174546; rs1535; rs102275 |
| rs1672692 | 11 | 113450819 | 0.00004276 | |
| rs9943540 | 11 | 127780902 | 0.00006544 | |
| rs11488811 | 11 | 50236215 | 0.00006825 | |
| rs10135535 | 14 | 76905866 | 0.00003968 | rs6637 |
| rs6564738 | 16 | 78727594 | 3.77E−06 | rs8057357; rs11150245; rs17726892; rs2016206 |
| rs35625 | 16 | 16077067 | 0.00009371 | |
| rs730547 | 17 | 30136219 | 0.00003572 | rs917443; rs2079664 |
| rs9952211 | 18 | 64952576 | 0.00005754 | rs11151487; rs951666 |
| rs17835885 | 19 | 57369983 | 0.00005541 | |
| rs6123762 | 20 | 56038754 | 0.00006658 | |
| rs4302309 | 22 | 26076350 | 0.00007323 | |
| rs10510608 | _3_{—} | _28280406_{—} | 0.00006922 | |

### EXAMPLE 2

### Candidate Gene Study to Identify SNPs Associated with BP Disease

A candidate gene approach was taken to identify loci associated with BP. The approach involved genotyping 466 bipolar cases and 465 controls for 1,727 SNPs located in 93 genes. The bipolar cases are from the NIMH Human Genetics Initiative's collection and the controls are ethnically matched NIMH control samples that have completed a psychiatric screen. The 93 genes were selected based on their association with bipolar disease, as well as their aberrant expression in our microarray experiments with human brain mRNA, and their implication in animal models with similar phenotypes. SNPs from Illumina's HumanHap550 arrays were selected that reside in regions 20 kb upstream and 10 kb downstream from each of the 93 candidate genes. The genotyped HumanHap550 chip covers a substantial fraction of the common genetic variation in individuals of European origin.

Genotyping was performed using the Infinium assay on Illumina's HumanHap550 arrays using 750 ng of genomic DNA extracted from transformed lymphoblasts. SNPs resided in regions 10 kb upstream and 5 kb downstream from each of the 93 candidate genes. Quality control samples included 15 trios and 24 replicate hybridizations. The overall quality of the Illumina genotyping data was excellent, yielding average call rates of 99.84% across all SNPs. The replication error rate was 1.5×10⁻⁵, and the error rate inferred from non-Mendelian inheritance was 2.5×10⁻⁴. Association tests were performed using logistic regression of each SNP genotype class against affected status. Three genetic models were tested (recessive, dominant, and multiplicative), and the minimum P-value of these tests was determined for each SNP. Gene-specific P-values were determined by correcting the minimum P-value for the number of SNPs in each gene and the degree of linkage disequilibrium across the gene.

Ten genes with p-values <0.05 were identified, significantly more than expected by chance (p=0.02). The lowest SNP-specific p-value was in the CAMKIIα gene (rs10515639), which had a p-value of 7×10⁻⁶ and an odds ratio of 1.6. The genes are presented in Table 2, below.

TABLE 2

| GENE | P Value | # SNPs | Top SNP | P Value |
|---|---|---|---|---|
| CAMKIIα | 0.0015 | 30 | rs10515639 | 2.6 × 10−5 |
| FGFR3 | 0.0047 | 1 | rs743682 | 0.0047 |
| GPR50 | 0.0086 | 7 | rs529386 | 0.0006 |
| CALB1 | 0.0119 | 3 | rs1805873 | 0.0060 |
| FZD7 | 0.0199 | 4 | rs2280509 | 0.0022 |
| NEUROG1 | 0.0222 | 1 | rs2344484 | 0.0098 |
| GAP43 | 0.0346 | 29 | rs9848541 | 0.0006 |
| AP3B2 | 0.0373 | 7 | rs4779041 | 0.0029 |
| COX7A1 | 0.0390 | 3 | rs753420 | 0.0058 |
| BDNF | 0.0403 | 8 | rs6265 | 0.0031 |

The SNPs in Table 2 are identified according to their ID Number (i.e., “rs### . . . ”) in the National Center for Biotechnology Information (NCBI) Single Nucleotide Polymorphism database (http://www.ncbi.nlm.nih.gov/projects/SNP/). The first p-value in the chart is the minimum p-value in the gene corrected for doing 3 tests (recessive, dominant, and multiplicative) and testing all the other SNPs in the gene, taking into account the linkage disequilibrium between them. The second is the minimum p-value in the gene.

### EXAMPLE 3

### Differential Exon Expression in Schizophrenia

The positive symptoms of schizophrenia can look like the symptoms in manic episodes, especially those with psychotic features (e.g., delusions of grandeur, hallucinations, disorganized speech, paranoia, etc.). The negative symptoms of schizophrenia can closely resemble the symptoms of a depressive episode (these include apathy, extreme emotional withdrawal, lack of affect, low energy, social isolation, etc.). Thus, objective molecular measurements that provide information relevant to diagnosis schizophrenia are very useful to clinicians and researchers. The following examples demonstrate how such measurements may be obtained.

Ten Affymetrix human exon arrays were hybridized with cDNA from five individual schizophrenia subjects and five unaffected family members. The experiment was repeated with the same samples and the data is presented as averages for each group (Schizophrenia and Controls). The plot for the transcript DSC2 (desmocollin) shows that 3 exons have no change in expression between schizophrenia and controls (FIG. 1). However, at the rest of the gene, which is not normally probed with Affymetrix U95 and U133 expression arrays, the exons show significant differences between schizophrenia and controls.

Table 3, below, shows the top 20 genes that were different when comparing lymphocyte exon expression of two families with schizophrenia to exon expression in unaffected family members.

TABLE 3

| | | p-value | | |
|---|---|---|---|---|
| Total | Affymetrix | (Diagnosis * | | |
| probes | Transcript | Exon ID) | RefSeq | Symbol |
| 89 | 3802980 | 1.07E−27 | NM_024422 | DSC2 |
| 149 | 3000342 | 1.67E−13 | NM_021116 | ADCY1 |
| 139 | 3854627 | 1.46E−12 | NM_000215 | JAK3 |
| 59 | 2746591 | 1.72E−11 | NM_001957 | EDNRA |
| 66 | 2765590 | 4.18E−11 | NM_139182 | CENTD1 |
| 8 | 3006572 | 1.12E−10 | NM_001013702 | LOC440258 |
| 21 | 2657665 | 1.49E−10 | NM_003722 | TP73L |
| 44 | 2927722 | 1.54E−10 | NM_014320 | HEBP2 |
| 41 | 3541383 | 2.10E−10 | NM_001172 | ARG2 |
| 29 | 2566848 | 2.50E−10 | NM_001025108 | AFF3 |
| 4 | 3802924 | 3.66E−10 | NM_001941 | DSC3 |
| 103 | 3577940 | 4.78E−10 | NM_024734 | CLMN |
| 157 | 3118651 | 6.99E−10 | NM_014957 | KIAA0870 |
| 61 | 2954678 | 1.37E−09 | NM_020750 | XPO5 |
| 106 | 2634965 | 2.08E−09 | NM_020235 | BBX |
| 89 | 3062868 | 2.18E−09 | NM_018842 | BAIAP2L1 |
| 12 | 2474341 | 2.81E−09 | NM_080592 | C2orf28 |
| 84 | 2360257 | 3.03E−09 | NM_000565 | IL6R |
| 108 | 2814642 | 3.48E−09 | NM_022132 | MCCC2 |
| 118 | 3320301 | 3.70E−09 | NM_014633 | SH2BP1 |

### EXAMPLE 4

### Allele-Specific Differences in Exon Expression and Relation to Schizophrenia

Using the same platform (i.e., the Affymetrix GeneChip® Human Exon 1.0 ST Array), gene expression data at the exon level of DPM2 was examined using regular non-SNP influenced probe sets, as well as exonic SNP information, taking advantage of the presence of around 2.2 million probe sets containing SNPs in the Affymetrix arrays. Because the Exon Array chip was not designed for SNP detection, the use of this information represents a new tool for obtaining information about functional variation (e.g., a non-synonymous coding SNP expressed in the disease state) in coding exons. This embodiment of the present invention can be used in the context of a Transmission Disequilibrium Test; familial, or case-control designs to study expression changes associated with a given disorder; identification of SNPs associated with that same disorder; and the interaction of gene variants and exon expression levels. In addition, the method can shed light into the genome wide interactomic differences associated with complex neuropsychiatric disorders, such as whether an SNP in the coding exon is associated with transcript alterations.

As a proof of principle, ten subjects were assayed on Human 1.0 ST Affymetrix Exon Arrays. Five schizophrenia probands and five related subjects were gender-matched relatives. Lymphocytes were transformed and cultured using standard conditions (Coriell Institute). Transformed lymphocytes were then harvested and processed for total RNA with Trizol extractions. The total RNA was reduced with ribo-minus procedure to eliminate ribosomal RNA, and labeling and hybridization according to Affymetrix HsExon array protocol.

Table 4 presents the exon expression data for DPM2 (dolichyl-phosphate mannosyltransferase polypeptide 2, regulatory subunit, Accession #NM_{—}003863.2).

TABLE 4

| | | | | | | | | | |
|---|---|---|---|---|---|---|---|---|---|
| 15 | 2 | 11 | 13 | 19 | 17 | 8 | 10 | 6 | 4 |

DPM2 probes

| | | | | | | | | | | |
|---|---|---|---|---|---|---|---|---|---|---|
| 3226238.1 | 1.60 | 3.32 | 0.65 | 2.15 | 2.69 | 0.89 | 3.56 | 1.45 | 1.91 | 1.17 |
| 3226238.2 | 5.02 | 0.90 | 2.63 | 1.29 | 5.18 | 1.59 | 1.12 | 2.31 | 1.69 | 5.20 |
| 3226238.3 | 2.21 | 3.06 | 2.63 | 3.30 | 1.31 | 0.99 | 0.90 | 1.07 | 1.48 | 1.80 |
| 3226238.4 | 1.49 | 1.08 | 5.05 | 2.40 | 2.54 | 1.86 | 1.51 | 1.71 | 1.91 | 1.47 |
| 3226239.1 | 7.67 | 7.21 | 7.88 | 8.22 | 6.89 | 7.52 | 6.76 | 7.78 | 8.01 | 7.80 |
| 3226239.2 | 7.49 | 7.09 | 7.03 | 7.87 | 6.89 | 6.88 | 6.93 | 8.02 | 7.14 | 7.58 |
| 3226239.3 | 7.33 | 7.07 | 7.60 | 8.42 | 6.98 | 7.49 | 7.08 | 8.07 | 7.45 | 7.81 |
| 3226240.1 | 9.56 | 9.42 | 9.67 | 10.58 | 9.45 | 9.44 | 9.51 | 9.70 | 9.38 | 10.37 |
| 3226240.2 | 8.84 | 8.75 | 9.23 | 9.75 | 7.90 | 8.96 | 8.67 | 8.77 | 8.86 | 9.86 |
| 3226240.3 | 6.48 | 6.14 | 6.32 | 7.06 | 5.10 | 5.64 | 6.49 | 7.32 | 5.53 | 6.44 |
| 3226240.4 | 8.91 | 7.84 | 8.57 | 9.35 | 8.56 | 8.63 | 8.23 | 8.95 | 8.72 | 9.04 |
| 3226241.1 | 1.09 | 1.84 | 3.49 | 5.33 | 5.50 | 6.13 | 7.08 | 7.20 | 7.54 | 8.40 |
| 3226241.2 | 1.60 | 2.23 | 3.08 | 1.48 | 1.68 | 7.65 | 7.26 | 8.90 | 8.67 | 9.41 |
| 3226241.3 | 2.48 | 1.49 | 2.78 | 5.96 | 2.84 | 7.23 | 7.59 | 7.59 | 7.62 | 9.22 |
| 3226241.4 | 1.95 | 1.28 | 1.31 | 1.20 | 0.88 | 6.93 | 6.76 | 8.02 | 8.02 | 8.76 |
| 3226242.1 | 5.60 | 5.07 | 6.41 | 6.57 | 6.35 | 5.93 | 5.52 | 6.86 | 6.43 | 7.01 |
| 3226242.2 | 5.62 | 6.10 | 5.56 | 5.76 | 5.01 | 4.58 | 2.56 | 4.35 | 6.20 | 2.97 |
| 3226242.3 | 5.74 | 5.12 | 4.71 | 3.53 | 4.20 | 3.90 | 4.44 | 4.35 | 5.44 | 5.42 |
| 3226242.4 | 3.85 | 4.37 | 5.59 | 4.27 | 3.94 | 4.85 | 5.88 | 5.18 | 3.74 | 5.52 |
| 3226243.1 | 9.37 | 8.73 | 8.93 | 9.70 | 8.52 | 8.83 | 9.04 | 8.65 | 8.89 | 9.56 |
| 3226243.2 | 9.36 | 9.04 | 9.67 | 10.20 | 9.43 | 9.13 | 9.01 | 9.51 | 9.70 | 10.26 |
| 3226243.3 | 10.25 | 9.59 | 10.00 | 10.60 | 9.65 | 8.71 | 9.81 | 9.65 | 9.76 | 10.94 |
| 3226243.4 | 9.03 | 9.08 | 8.90 | 9.84 | 8.67 | 8.91 | 8.60 | 8.54 | 8.83 | 9.71 |
| 3226244.1 | 9.46 | 8.15 | 9.27 | 9.21 | 8.62 | 8.47 | 8.58 | 9.83 | 9.09 | 8.74 |
| 3226244.2 | 7.11 | 6.70 | 6.09 | 6.84 | 6.36 | 5.46 | 5.99 | 6.05 | 5.98 | 5.45 |
| 3226244.3 | 7.47 | 7.12 | 7.30 | 7.65 | 6.18 | 7.26 | 6.96 | 7.42 | 6.36 | 7.92 |
| 3226244.4 | 5.57 | 4.06 | 5.33 | 4.92 | 3.94 | 4.45 | 1.12 | 4.66 | 4.27 | 6.00 |
| 3226245.1 | 7.36 | 6.45 | 7.27 | 8.00 | 7.40 | 6.69 | 7.41 | 7.49 | 7.48 | 8.01 |
| 3226245.2 | 5.65 | 4.06 | 6.62 | 6.87 | 5.89 | 5.73 | 5.38 | 5.84 | 5.18 | 6.68 |
| 3226245.3 | 6.89 | 7.04 | 7.15 | 7.54 | 7.05 | 5.86 | 6.72 | 7.28 | 6.43 | 7.01 |
| 3226245.4 | 6.63 | 6.04 | 6.44 | 6.27 | 6.62 | 5.58 | 6.24 | 6.76 | 6.79 | 7.03 |
| 3226246.1 | 7.22 | 6.65 | 7.39 | 8.09 | 7.83 | 8.01 | 8.03 | 7.90 | 7.96 | 7.98 |
| 3226246.2 | 7.06 | 6.72 | 7.63 | 7.26 | 6.53 | 5.88 | 6.87 | 7.09 | 7.06 | 7.55 |
| 3226246.3 | 6.33 | 5.48 | 6.09 | 5.10 | 4.77 | 5.04 | 6.28 | 6.80 | 6.40 | 5.72 |
| 3226246.4 | 1.28 | 0.90 | 1.07 | 2.28 | 2.24 | 1.86 | 1.79 | 3.11 | 1.29 | 4.07 |
| 3226247.1 | 8.57 | 8.42 | 8.74 | 8.80 | 8.40 | 8.92 | 8.80 | 9.38 | 8.50 | 8.74 |
| 3226247.2 | 8.45 | 8.36 | 8.52 | 9.04 | 8.03 | 7.63 | 8.19 | 8.46 | 8.97 | 9.12 |
| 3226247.3 | 8.11 | 8.45 | 8.66 | 8.94 | 7.93 | 7.83 | 7.36 | 8.37 | 8.34 | 9.28 |
| 3226247.4 | 8.08 | 8.24 | 8.83 | 9.04 | 8.09 | 7.40 | 8.45 | 8.65 | 8.03 | 8.95 |
| 3226248.1 | 6.38 | 5.57 | 6.92 | 6.56 | 5.30 | 5.67 | 6.12 | 6.77 | 6.31 | 6.13 |
| 3226248.2 | 4.77 | 3.97 | 4.71 | 4.60 | 3.94 | 2.01 | 4.44 | 5.40 | 5.40 | 5.80 |
| 3226248.3 | 7.22 | 2.79 | 6.27 | 7.06 | 5.18 | 5.67 | 6.75 | 6.75 | 6.69 | 6.10 |
| 3226248.4 | 5.50 | 4.22 | 5.78 | 6.81 | 5.98 | 4.75 | 4.89 | 7.21 | 5.79 | 6.02 |
| 3226249.1 | 4.66 | 5.75 | 5.36 | 6.03 | 4.60 | 5.28 | 4.69 | 5.74 | 6.22 | 6.13 |
| 3226249.2 | 5.65 | 3.19 | 5.01 | 6.22 | 5.33 | 4.90 | 4.99 | 5.82 | 3.04 | 5.28 |
| 3226249.3 | 7.78 | 6.88 | 6.74 | 7.52 | 6.24 | 7.30 | 8.11 | 7.59 | 6.56 | 7.84 |
| 3226249.4 | 7.74 | 6.67 | 7.47 | 7.62 | 7.32 | 5.61 | 7.66 | 7.70 | 6.66 | 7.18 |
| 3226250.1 | 7.56 | 7.37 | 5.65 | 5.68 | 5.84 | 5.70 | 5.12 | 5.59 | 5.40 | 6.54 |
| 3226250.2 | 8.23 | 8.42 | 7.46 | 8.03 | 7.43 | 7.75 | 8.27 | 8.06 | 8.45 | 8.24 |
| 3226250.3 | 2.75 | 1.97 | 2.94 | 2.40 | 2.10 | 2.31 | 2.88 | 3.00 | 3.74 | 2.56 |
| 3226250.4 | 1.18 | 1.38 | 3.08 | 0.95 | 1.08 | 0.89 | 1.12 | 1.71 | 1.11 | 1.00 |
| 3226251.1 | 1.95 | 2.37 | 2.00 | 1.69 | 1.42 | 2.77 | 2.56 | 3.95 | 2.40 | 3.58 |
| 3226251.2 | 1.71 | 2.93 | 4.10 | 2.28 | 2.54 | 1.46 | 4.30 | 1.45 | 3.29 | 2.83 |
| 3226251.3 | 4.97 | 3.78 | 6.09 | 5.50 | 4.77 | 5.83 | 4.99 | 5.82 | 5.14 | 5.24 |
| 3226251.4 | 5.06 | 5.97 | 5.17 | 5.47 | 5.33 | 5.73 | 5.28 | 4.82 | 6.79 | 5.72 |
| 3226252.1 | 5.53 | 5.24 | 5.98 | 5.26 | 5.59 | 5.00 | 5.72 | 6.24 | 6.56 | 4.69 |
| 3226252.2 | 6.07 | 4.56 | 4.47 | 4.20 | 2.84 | 2.31 | 3.68 | 5.37 | 4.48 | 4.23 |
| 3226252.3 | 2.08 | 1.49 | 5.81 | 4.97 | 5.65 | 6.25 | 5.03 | 5.62 | 5.76 | 5.72 |
| 3226252.4 | 5.65 | 5.03 | 6.41 | 6.29 | 4.35 | 5.49 | 5.24 | 5.98 | 5.18 | 6.46 |

DPM2 Exon Expression in 10 Lymphocyte samples shows high inter-individual variation in probes containing SNP (shown in gray). Numbers at the top of each column identify the different subjects from whom samples were taken and analzyed. The units are intensity log2 scale, so that the difference between subject 15 and 4 is 2\hat{ }(8.4 − 1.0) = 168.9 fold change.

The log2 scale for the average probeset expression levels across the DPM2 gene are shown. The range of expression may exceed 100 fold, for example, in probe 322624.1, which shows a minimum in subject 15 to a maximum in subject 4 in the DPM2 gene. This range of individual differences might not be seen in whole transcript analysis when averaging all of the exons or when looking only at a single exon in each transcript. The experiment was repeated and the same expression variability was seen in 8 unrelated controls.

The variability in DPM2 exon expression observed in this example is due to genetic sequence variation within the exon that causes the transcripts to differentially hybridize to the Affymetrix probes (Table 5).

TABLE 5

| Affymetrix ST Human Exon | (Number of |
|---|---|
| 1.0 Target Sequence | Mismatches due |
| For DPM2 Probes | to SNPs in probe) |
| GTCTTCAGCATCACATAGGAGATGA | Probe 1 (2) |
| TCTTCAGCATCACATAGGAGATGAA | Probe 2 (1) |
| TCAGCATCACATAGGAGATGAACAG | Probe 3 (1) |
| AGCATCACATAGGAGATGAACAGTC | Probe 4 (1) |
| GTCTTCAGCATCACATAGGAGATGAACAGTC | Consensus |
| GACTGTTCATCTCCTATGTGATGCTGAAGAC | Actual sequence (−) |

The Table shows the consequence of including a SNP in the probe design. The hybridization will be significantly reduced in any individuals with the minor allele. This algorithm will help to greatly reduce the identification of alternative splicing events, when the real underlying biological event is detection or lack of detection of an exonic SNP.

All the individuals with low expression of the DPM2 exon IV were homozygous for the minor alleles of SNPs rs6781 (T/C) and rs7997 (C/G), which is a non-synonymous coding SNP. These genotypes were confirmed by sequencing on an ABI DNA sequencer.

The allelic and exon quantitative PCR measures of cDNA from each individual were analyzed and correlated with genotype. These results are shown in Table 5 and Table 6.

TABLE 6

| Genotype | Affy probe | 7997-C | 7997-G | Exon4 |
|---|---|---|---|---|
| GG | 2.68 | 0.00 | 1.63 | 1.72 |
| GG | 1.76 | 0.00 | 1.48 | 1.38 |
| GG | 2.80 | 0.00 | 1.41 | 1.07 |
| CG | 0.70 | 0.49 | 0.60 | 0.96 |
| CG | 1.66 | 0.39 | 0.44 | 0.78 |
| CC | 0.03 | 2.15 | 0.00 | 1.59 |
| CC | 0.07 | 0.72 | 0.00 | 1.23 |
| CC | 0.06 | 0.70 | 0.00 | 0.80 |
| CC | 0.10 | 0.89 | 0.00 | 0.83 |
| CC | 0.15 | 1.00 | 0.00 | 0.91 |

The Table shows the genotype (column 1), expression levels of the DPM2 gene from the Human 1.0 ST Affymetrix Exon Array (column 2), and allelic specific RT-PCR as a function of the observed genotypes (column 3, 4, and 5). Exon4 expression levels correspond to the real expression of exon 4 as measured with primers not affected by the SNPs. Note that the Affymetrix probe contains SNPs and therefore hybridization using that probe does not detect the actual exon 4 levels, as shown in column 4.

The expression levels reported by the Affymetrix probe correspond closely to the expression observed in the allelic specific RT-PCR experiment with the allele G matching. This example shows that the expression levels reported using the exon array correspond to a genotyping effect and can discriminate efficiently between the three possible genotypes (GG, GC and CC). Thus, GG subjects have the highest expression levels, GC subjects have intermediate levels and CC subjects show an almost complete absence of expression when an Affymetrix probe that contains a SNP is used. This experimental result was confirmed using an additional eight unrelated control samples.

Thus, the results shown here with DPM2 show that the use of probe sets containing SNPs in the Affymetrix GeneChip® Human Exon 1.0 ST Array allows genotypes to be accurately inferred from exon array data when a combination of normalization algorithms, quality control and data processing are used. It is estimated that about 100,000 SNP genotype calls can be made accurately using cDNA samples.

The DPM2 example also demonstrates how to measure correct allelic expression. The solution is to allow incorporation of SNP calls into an exon array, then use those probes for accurate calls of expression and genotypes. Some probes with SNPs might be used for genotype calls, and the remaining probes can be used for exon expression summarization. Various genetic models of fit between SNP calls and non-SNP-containing probeset expression can be tested to identify dominant or additive effects. Functional variations can be determined in one experiment and run in any tissue sample from which RNA could be extracted. As a test for the correct SNP genotype on this platform, running individual DNA on the a slightly modified protocol will allow accurate determination of whether a true genotype call is being made, as opposed to a hybridization artifact. Thus, another use of this method is to compare DNA and cDNA from an individual to determine whether a specific allele is being expressed.

In a second test using DSC2 (RefSeq Accession #NM_{—}024422), the presence of the SNP rs12954874 affects only one out of 4 probes, i.e., Probe 1 (Table 7). The presence of the T allele results in an apparent increase of the expression of the exon, as measured by the four different microarray probes and by the allele specific RT-PCR.

TABLE 7

| | | | | | | | Probe |
|---|---|---|---|---|---|---|---|
| Genotype | 4874 | 4874-C | 4874-T | Probe 1 | Probe 2 | Probe 3 | 4 |
| CC | 1.7 | 1.6 | 0.1 | 104.0 | 44.0 | 20.0 | 57.0 |
| CC | 1.1 | 1.0 | 0.1 | 138.6 | 2.8 | 58.6 | 40.6 |
| CC | 1.3 | 1.5 | 0.1 | 189.7 | 9.7 | 36.7 | 67.7 |
| CC | 11.0 | 9.9 | 0.6 | 479.2 | 79.2 | 97.2 | 185.2 |
| CC | 0.2 | 0.3 | 0.0 | 242.4 | 58.4 | 39.4 | 98.4 |
| CC | 0.1 | 0.1 | 0.0 | 62.2 | 2.2 | 59.2 | 5.3 |
| CC | 0.1 | 0.2 | 0.0 | 64.8 | 13.8 | 10.0 | 16.8 |
| CC | 3.1 | 5.3 | 0.5 | 642.2 | 136.2 | 111.2 | 237.2 |
| CT | 1.1 | 0.6 | 0.7 | 226.9 | 25.9 | 84.9 | 39.9 |
| CT | 23.8 | 12.7 | 14.4 | 1280.8 | 357.8 | 259.8 | 597.8 |

The Table shows the expression levels of the DSC2 gene from the Human 1.0 ST Affymetrix Exon Array allelic specific RT-PCR as a function of the observed genotypes. Expression levels in column 4874 correspond to the real expression of the exon as measured with primers not affected by the SNPs. Probes 1 to 4 correspond to the expression levels of the four probes for that exon in the exon array; only probe 1 is directly affected by the SNP.

In summary, the present invention provides at least 4 different methods for using the same data derived from one sample hybridization on one chip to detect (1) exon-specific gene expression; (2) exonic SNPs genotypes; (3) the interaction of the exonic SNPs investigated and the exon-specific expression levels; and (4) allele-specific gene expression.

Because the exon-specific expression levels and the allele-specific expression levels are derived from the same RNA sample-experiment, the normalization is simplified and the variation is highly reduced. In contrast to the methods described here, the prior art relies on using separate platforms and does not anticipate that using probes containing SNPs and probes not containing SNPs in the Affymetrix exon arrays would be useful across multiple exons to determine a functional haplotype relationship. The present invention provides a general method allowing to diagnose and study neuropsychiatric disorders using one unique platform, the Affymetrix GeneChip® Human Exon 1.0 ST Array.

The above examples are provided to illustrate the invention but not to limit its scope. Other variants of the invention will be readily apparent to one of ordinary skill in the art and are encompassed by the appended claims. All publications, databases, Genbank sequences, patents, and patent applications cited herein are hereby incorporated by reference.

What is claimed is:

1. A method for diagnosing or identifying a human subject having an increased risk of bipolar disorder, the method comprising: obtaining a sample from the subject; and b) analyzing said sample for the occurrence of at least one single nucleotide polymorphism (SNP) selected from the SNPs listed in Table 1 and Table 2, wherein an occurrence of at least one of said SNPs is associated with increased risk of developing bipolar disorder; and recording or reporting the diagnosis or risk assessment.

2. A method for diagnosing bipolar disorder in a human subject, comprising: obtaining a sample from the subject; identifying an occurrence of a single nucleotide polymorphism (SNP) in linkage disequilibrium with one or more SNPs or genes selected from the SNPs or genes listed in Table 1 and Table 2, wherein an occurrence of one or more SNPs in linkage disequilibrium with an SNP or gene of Table 1 or Table 2 is associated with an increased likelihood that the patient is suffering from bipolar disorder; and reporting or recording said diagnosis based on said occurrence.

3. A method for diagnosing the presence of a polymorphism in a human gene selected from the list of genes in Table 2, wherein said polymorphism predisposes said human to bipolar disease, said method comprising: obtaining a sample from a human subject; contacting said sample with a reagent, wherein said reagent provides a detectable signal indicative of the presence of a polymorphism in said gene; and reporting or recording said diagnosis based on said signal.

4. The method of claim 3, wherein said polymorphism is selected from the polymorphisms listed in Table 1 and Table 2.

5. The method of claim 3, wherein said polymorphism is in linkage disequilibrium with a polymorphism listed in Table 1 or Table 2.

6. A method for distinguishing between bipolar illness and schizophrenia in a subject, comprising (a) obtaining a sample from said subject; (b) identifying an occurrence of one or more single nucleotide polymorphisms (SNPs) selected from the SNPs listed in Table 1 and Table 2, wherein an occurrence of one or more of said SNPs is associated with an increased likelihood of bipolar disorder; and (c) reporting or recording a likelihood of bipolar disorder or schizophrenia based on said identification.

7. The method of claim 6, further comprising measuring expression of one or more genes selected from the genes listed in Table 3.

8. The method of any of claims 1-7, further comprising treating said subject to alleviate one or more symptoms of bipolar illness.

9. A method for identifying a human subject with an increased likelihood of schizophrenia, comprising obtaining a sample from said subject; analyzing said sample for the expression of one or more of the exons of the genes listed in Table 3; correlating a significant difference in exon expression relative to a control with an increased likelihood of schizophrenia; and reporting or recording said conclusion with respect to said increased likelihood of schizophrenia.

10. The method of claim 9, wherein said exon is selected from the group consisting of the differentially expressed DSC2 exons, as shown in FIG. 1 and Table 7, and the differentially expressed DPM2 exon shown in Table 4 and Table 6.

11. The method of claim 9, wherein an SNP selected from the group consisting of SNPs rs6781 and rs7997 in exon IV of the DPM2 gene is identified in said subject; and wherein said SNP is correlated with the levels of expression of said exon.

12. The method of claim 9, wherein SNP rs12954874 in exon II of the DSC2 gene is identified in said subject; and wherein said SNP is correlated with the levels of expression of said exon.


SEQUENCE LISTING (7 sequences)

SEQ ID NO: 1 - protein, 200 aa, Artificial Sequence
  Description of Artificial Sequencepoly-Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 2 - DNA, 25 bp, Artificial Sequence
  Description of Artificial Sequencedolichyl-phosphate mannosyltransferase polypeptide 2, regulatory subunit (DPM2), Probe 1
gtcttcagcatcacataggagatga

SEQ ID NO: 3 - DNA, 25 bp, Artificial Sequence
  Description of Artificial Sequencedolichyl-phosphate mannosyltransferase polypeptide 2, regulatory subunit (DPM2), Probe 2
tcttcagcatcacataggagatgaa

SEQ ID NO: 4 - DNA, 25 bp, Artificial Sequence
  Description of Artificial Sequencedolichyl-phosphate mannosyltransferase polypeptide 2, regulatory subunit (DPM2), Probe 3
tcagcatcacataggagatgaacag

SEQ ID NO: 5 - DNA, 25 bp, Artificial Sequence
  Description of Artificial Sequencedolichyl-phosphate mannosyltransferase polypeptide 2, regulatory subunit (DPM2), Probe 4
agcatcacataggagatgaacagtc

SEQ ID NO: 6 - DNA, 31 bp, Artificial Sequence
  Description of Artificial Sequencedolichyl-phosphate mannosyltransferase polypeptide 2, regulatory subunit (DPM2), consensus target sequence
gtcttcagcatcacataggagatgaacagtc

SEQ ID NO: 7 - DNA, 31 bp, Artificial Sequence
  Description of Artificial Sequencedolichyl-phosphate mannosyltransferase polypeptide 2, regulatory subunit (DPM2), actual target sequence
gactgttcatctcctatgtgatgctgaagac